(19) United States
(12) Patent Application Publication
(10) Pub. No.: US 2026/0224427 A1
(43) Pub. Date: Aug. 6, 2026

(54) SYSTEMS, METHODS AND APPARATUS FOR AUGMENTED MOBILITY

(71) Applicant: Dephy, Inc., Boxborough, MA (US)

(72) Inventors: Luke Mooney, Sudbury, MA (US); William Marvin, Canton, MA (US); Jonathan Cummings, Concord, MA (US)

(73) Assignee: Dephy, Inc., Boxborough, MA (US)

(21) Appl. No.: 19/149,831

(22) PCT Filed: Jan. 22, 2024
(86) PCT No.: PCT/US2024/012350

§ 371 Date: Jul. 21, 2025

Related U.S. Application Data
(60) Provisional application No. 63/440,483, filed on Jan. 23, 2023.

(51) Int. Cl. A61H 3/00; A61H 1/02

(52) U.S. Cl. CPC A61H 3/00; A61H 1/0266; A61H 2003/007; A61H 2201/1642; A61H 2201/165; A61H 2201/1676

(57) ABSTRACT

Systems, methods and apparatus for augmented mobility are provided. An exoskeleton can include foot plate. The exoskeleton can include a first joint located at an end of the foot plate. The first joint can define a first axis. The exoskeleton can include a second joint mechanically coupled at a side of the foot plate. The second joint can define a second axis. The exoskeleton can include a linkage member coupled with the first joint and the second joint. The exoskeleton can include a structural member extending from the second joint and along a lower limb of the user. The exoskeleton can include a brace member mechanically coupled with the structural member. The brace member can couple the exoskeleton with the lower limb. The exoskeleton can include an actuator system to exert a force between the linkage member and the structural member to augment motion of the user.

### CROSS-REFERENCE TO RELATED APPLICATION

This application claims priority to U.S. Provisional Application No. 63/440,483, filed Jan. 23, 2023, which is incorporated herein by reference in its entirety.

### BACKGROUND

Exoskeletons can be worn by a user to facility movement of limbs of the user.

### SUMMARY

This technology is directed to systems, methods and apparatus for augmented mobility. More specifically, this disclosure is directed to using artificial structures and actuators (e.g., an exoskeleton) to augment a user's activities by providing mechanical power directly to the user's body. Conventional artificial structures and actuators can unnecessarily limit normal movement of a user's body and can cause discomfort and skin irritations. The present disclosure is directed to an exoskeleton configured to provide the augmented movement and eliminate or reduce unnecessarily restricting movement of the user and prevent or reduce unwanted irritations. For example, the exoskeleton can include joints that align with biological joints of the user to reduce relative motion at the interface between the exoskeleton and the user. Limiting such relative motion can reduce any skin irritations caused between the exoskeleton and the skin of the user. The exoskeleton can include a unidirectional actuator system to provide augmented motion in a single direction and reduce resistance to user motion in other directions. The exoskeleton can integrate the electro-mechanical components needed to provide the augmented motion into the other components of the exoskeleton to reduce the overall size and bulkiness of the exoskeleton, making it easier to use and more comfortable for the user.

At least one aspect is directed to an exoskeleton. The exoskeleton can include a first joint located at an end of the foot plate. The first joint can define a first axis and be configured to be proximate to or behind a heel of a user. The exoskeleton can include a second joint mechanically coupled at a side of the foot plate. The second joint can define a second axis. The exoskeleton can include a linkage member coupled with, and extending between, the first joint and the second joint. The exoskeleton can include a structural member extending from the second joint. The structural member can extend along a lower limb of the user. The exoskeleton can include a brace member mechanically coupled with the structural member. The brace member can couple the exoskeleton with the lower limb. The exoskeleton can include an actuator system coupled with the structural member. The actuator system can exert a force between the linkage member and the structural member to augment motion of the user.

At least one aspect is directed to a method. The method can include positioning a first joint at an end of a foot plate. The first joint can define a first axis and be configured to be proximate to a heel of a user. The method can include coupling a second joint at a side of the foot plate. The second joint can define a second axis. The method can include coupling the first joint with the second joint via a linkage member. The method can include coupling a structural member with the second joint. The structural member can extend along a lower limb of the user. The structural member can include a brace member to couple the exoskeleton with the lower limb. The method can include coupling an actuator system with the structural member. The actuator system can exert a force between the linkage member and the structural member to augment motion of the user.

At least one aspect is directed to a system configured to augment movement of a user. The system can include an exoskeleton. The exoskeleton can include a foot plate. The exoskeleton can include a first joint located at an end of the foot plate. The first joint can define a first axis and be configured to be proximate to a heel of a user. The exoskeleton can include a second joint mechanically coupled at a side of the foot plate. The second joint can define a second axis. The exoskeleton can include a linkage member coupled with, and extending between, the first joint and the second joint. The exoskeleton can include a structural member extending from the second joint. The structural member can extend along a lower limb of the user. The exoskeleton can include a brace member mechanically coupled with the structural member. The brace member can couple the exoskeleton with the lower limb. The exoskeleton can include an actuator system coupled with the structural member. The actuator system can exert a force between the linkage member and the structural member to augment motion of the user.

At least one aspect is directed to a method for providing an exoskeleton. The method includes providing the exoskeleton. The exoskeleton can include a foot plate. The exoskeleton can include a first joint located at an end of the foot plate. The first joint can define a first axis and be configured to be proximate to a heel of a user. The exoskeleton can include a second joint mechanically coupled at a side of the foot plate. The second joint can define a second axis. The exoskeleton can include a linkage member coupled with, and extending between, the first joint and the second joint. The exoskeleton can include a structural member extending from the second joint. The structural member can extend along a lower limb of the user. The exoskeleton can include a brace member mechanically coupled with the structural member. The brace member can couple the exoskeleton with the lower limb. The exoskeleton can include an actuator system coupled with the structural member. The actuator system can exert a force between the linkage member and the structural member to augment motion of the user.

### BRIEF DESCRIPTION OF THE DRAWINGS

The details of one or more implementations of the subject matter described in this specification are set forth in the accompanying drawings and the description below. Other features, aspects, and advantages of the subject matter will become apparent from the description, the drawings, and the claims.

FIG. 1 illustrates skeletal views of an example lower limb, in accordance with some aspects.

FIG. 2 illustrates a front perspective view of an example exoskeleton, in accordance with some aspects.

FIG. 3 illustrates a front perspective view of a linkage portion, in accordance with some aspects.

FIG. 4 illustrates an inverted front perspective view of an example linkage portion, in accordance with some aspects.

FIG. 5A illustrates a side view of an example foot with a projection of an ST joint axis of rotation, in accordance with some aspects.

FIG. 5B illustrates a top front view of an example foot with a projection of a TC joint axis of rotation, in accordance with some aspects.

FIG. 6 illustrates a rear view of an example exoskeleton, in accordance with some aspects.

FIG. 7 illustrates a side view of an example ankle model, in accordance with some aspects.

FIG. 8 illustrates a side view of an example exoskeleton, in accordance with some aspects.

FIG. 9A illustrates a side perspective view of an example exoskeleton, in accordance with some aspects.

FIG. 9B illustrates a side perspective view of an example exoskeleton, in accordance with some aspects.

FIG. 9C illustrates a side perspective view of an example exoskeleton, in accordance with some aspects.

FIG. 10A illustrates a front and rear view of an example exoskeleton, in accordance with some aspects.

FIG. 10B illustrates a front and rear view of an example exoskeleton, in accordance with some aspects.

FIG. 11 illustrates a front perspective view of an example exoskeleton, in accordance with some aspects.

FIG. 12 illustrates a front perspective view of an example exoskeleton, in accordance with some aspects.

FIG. 13 illustrates a front perspective view of an example exoskeleton, in accordance with some aspects.

FIG. 14 illustrates a front perspective view of an example exoskeleton, in accordance with some aspects.

FIG. 15 illustrates a front perspective view of an example exoskeleton, in accordance with some aspects.

FIG. 16 illustrates a rear view of an example exoskeleton, in accordance with some aspects.

FIG. 17 illustrates a rear view of an example foot and ankle, in accordance with some aspects.

FIG. 18 illustrates a rear view of an example foot and ankle, in accordance with some aspects.

FIG. 19 illustrates a rear view of an example foot and ankle, in accordance with some aspects.

FIG. 20 illustrates a top view of an example foot and ankle, in accordance with some aspects.

FIG. 21 illustrates a side view of an example foot and ankle, in accordance with some aspects.

FIG. 22 illustrates a side view of an example exoskeleton, in accordance with some aspects.

FIG. 23 illustrates a side view of an example exoskeleton, in accordance with some aspects.

FIG. 24 illustrates a rear perspective view of an example exoskeleton, in accordance with some aspects.

FIG. 25 illustrates a rear exploded view of an example exoskeleton, in accordance with some aspects.

FIG. 26 illustrates a rear perspective view of an example exoskeleton, in accordance with some aspects.

FIG. 27A illustrates a side view of an example exoskeleton, in accordance with some aspects.

FIG. 27B illustrates a rear view of an example exoskeleton, in accordance with some aspects.

FIG. 28 illustrates a rear view of an example exoskeleton, in accordance with some aspects.

FIG. 29 illustrates a rear view of an example exoskeleton, in accordance with some aspects.

FIG. 30 illustrates side views of an example exoskeleton, in accordance with some aspects.

FIG. 31 illustrates a perspective view of an example winch roller, in accordance with some aspects.

FIG. 32 illustrates perspective views of example tensile elements, in accordance with some aspects.

FIG. 33 illustrates a side view of an example exoskeleton, in accordance with some aspects.

FIG. 34 illustrates a side view of an example exoskeleton, in accordance with some aspects.

FIG. 35 illustrates a flow diagram of a method for assembling an example exoskeleton, in accordance with some aspects.

FIG. 36 illustrates a flow diagram of a method for providing an example exoskeleton, in accordance with some aspects.

Like reference numbers and designations in the various drawings can indicate like elements.

### DETAILED DESCRIPTION

This disclosure relates generally to performance enhancing wearable technologies. Particularly, this disclosure relates to apparatuses, systems, and methods for augmented mobility (e.g., augmented ankle movement). An exoskeleton (also referred to as a foot-ankle exoskeleton) can augment a user's activities by using artificial structures and actuators to provide mechanical power directly to the user's body. However, the exoskeleton can have the challenge of exerting forces on the human body without limiting normal movement. Exoskeletons can have rigid components to react to internal structural forces so that the device can apply beneficial moments about biological joints. An exoskeleton with artificial joints that are perfectly aligned with the human body can have no (or minimal—e.g., 10 mm) relative movement between the device and the body during movement. However, if the artificial joints are misaligned with the biological joints and there are no other compensatory mechanisms, there can be relative movement at the interface between the human and device, which can result in abrasions, sores, cuts, or hot spots on the user's skin.

A functioning ankle exoskeleton can include some degree of rigid mechanical structure around the human foot and ankle to transmit and manage the force loads required to produce augmentation. The human ankle has multiple degrees of freedom that must be in alignment with any mechanical structure surrounding it specifically, the talocrural joint enabling plantarflexion and dorsiflexion and the subtalar joint responsible for inversion and eversion.

The subtalar joint can be difficult to functionally integrate into the structure of an exoskeleton because the natural axis of rotation of the joint projects through the foot itself, thus natural alignment is challenging when designing a structure around the foot that cannot interfere with, constrain movement, or cause discomfort. One solution can include disposing the subtalar joint on the lateral side of the exoskeleton structure immediately below the talocrural joint.

Because the axis of rotation for the subtalar joint is misaligned, the distance of the misalignment is translated up the ankle with each step. This resulting shear force can be mitigated by allowing the human attachment point on the upper leg to have a degree of freedom. With this solution, the more massive components of the exoskeleton such as the motor, battery, PCB and transmission components can move freely around the leg generating inertia and limiting attachment solutions. Moving the subtalar joint anterior to the foot and centered along the leg, on the frontal plane can mitigate this misalignment.

The technical solutions described herein can have the technical advantage of better aligning an exoskeleton's artificial joints with a user's biological joints to reduce relative motion at the interface while providing meaningful augmentation. The technical solutions described herein can include at least one ankle exoskeleton that includes a winch actuator. The winch actuator can have a high power density, can have a compact form factor, and can be mechanically simple, which can allow the winch actuator to be located posterior or anterior to the leg for more direct power transmission and more favorable mass distribution. The ankle exoskeleton can include a symmetrical design with lateral and medial structures to oppose the winch and span the talocrural (TC) and subtabular (ST) joints. The symmetrical design can facilitate more efficient power transmission as compared to lateral only devices, can provide the opportunity to explore more minimal structures to oppose compression forces of the winch actuator, and facilitate a more favorable distribution of mass. The symmetrical design can distribute force loads over a greater area to allow for less rigid and bulky materials and design configurations by judicious placement of rigid material specifically along the axis of compression and utilizing soft-good materials strong in tensile strength to constrain the foot and comfortably react against the shank. Placing the heaviest components of the exoskeleton symmetrically and posterior to the leg can correct the mass imbalance and rotational inertia that can be present with various lateral exoskeleton configurations. The ankle exoskeleton can be more naturally aligned (close to symmetrical) with a ST joint. The alignment between artificial joints and biological joints can reduce the translative movement between the interface between the joints, which can reduce the discomfort of a user and reduce the skin irritation and injuries that can occur. Furthermore, with the translative effect mitigated, the mass around the leg can be securely attached and held stationary to a leg further reducing inertial effects felt on the leg. Further integration of the electro-mechanical components of the exoskeleton, such as batteries or PCB boards, can be integrated into soft-good structures around the leg, which can reduce bulk and rigid mass. These structures can be optimized for attributes such as lower mass, less rigid parts, better mass distribution, more comfortable at high force loads, and ultimately a higher preforming device.

### I. Exoskeleton Overview

Exoskeletons (e.g., lower limb exoskeleton, knee exoskeleton, back exoskeleton, etc.) can include devices worn by a person to augment physical abilities. Exoskeletons can be considered passive (e.g., not requiring an energy source such as a battery) or active (e.g., requiring an energy source to power electronics and usually one or many actuators). Exoskeletons may be capable of providing large amounts of force, torque and/or power to the human body in order to assist with motion.

Exoskeletons can transfer energy to the human and may not interfere with the natural range of motion of the body. Exoskeletons can convert the energy source into useful mechanical force, torque, or power. Onboard electronics (e.g., controllers) can control the exoskeleton. Output force and torque sensors can also be used to make controlling easier.

### II. Ankle Exoskeleton

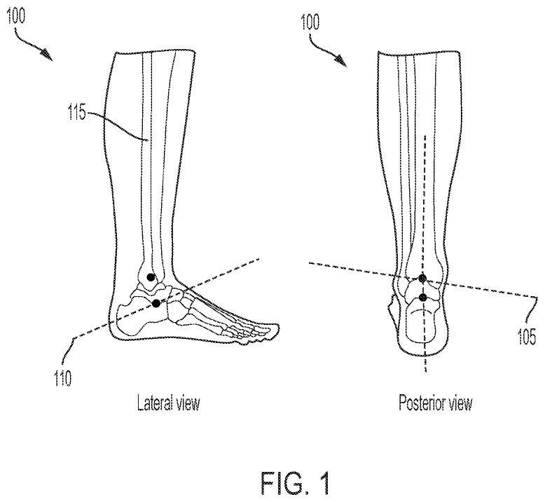

FIG. 1 illustrates a skeletal view of an example lower limb 100. The lower limb 100 can include a talocrural (TC) joint 105. The TC joint 105 is a physiological joint that enables plantarflexion and dorsiflexion. The lower limb 100 can include a subtalar (ST) joint 110. The ST joint 110 is a physiological joint that enables eversion and inversion. The lower limb 100 can include a shank 115. The shank 115 is the portion of the body between the ankle joint and the knee joint.

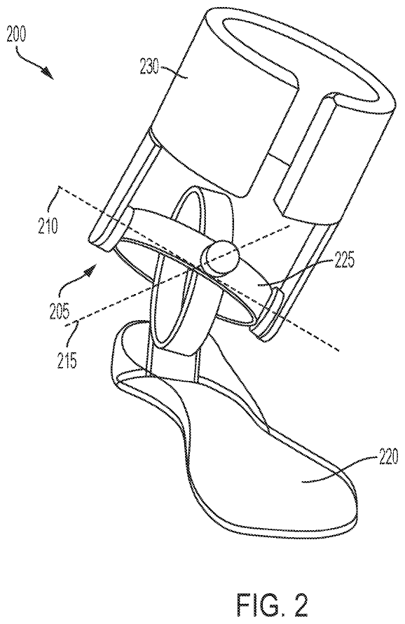

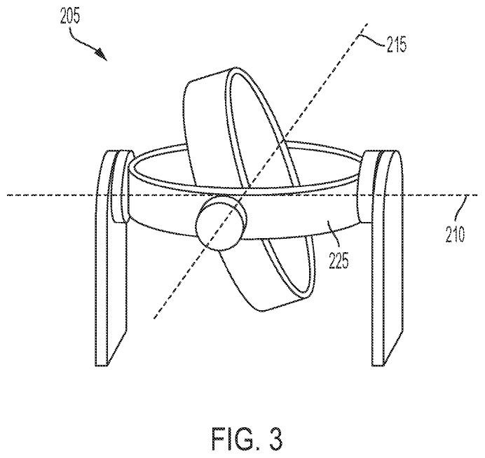

FIGS. 2-6 illustrate an exoskeleton 200. The exoskeleton 200 can include three basic structures that are connected along two axes. The exoskeleton 200 can include a shank structure that can be designed to be affixed to the human shank 115. The exoskeleton 200 can include a foot structure that can be designed to be affixed to the human foot. The exoskeleton 200 can include a ST-TC link structure that can be connected to the foot structure along an exoskeleton ST axis and can be connected to the shank structure along an exoskeleton TC axis. These joint connections may occur at one or more joints.

The exoskeleton TC axis and the exoskeleton ST axis can be designed to align closely with the user's ST and TC axis. However, certain approximations and simplifications can be made to ensure manufacturability, safety, and proper torque application. For example, an exoskeleton TC Joint and an exoskeleton ST Joint can be orthogonal to each other. This ensures that an augmentation torque applied across the exoskeleton TC joint does not result in any torque being applied to the exoskeleton ST joint. If these two joints are not orthogonal, as is the case for the biological axes, a torque applied to one can result in unintended torques in the other. For example, it is typically favorable to apply only a plantarflexing/dorsiflexing torque without applying an everting/inverting torque.

It may also be advantageous to modify the exoskeleton axes locations from their respective biological locations. The exoskeleton ST joint can be moved upwards to reduce the posterior protrusion that can impact heel strike and cause a tripping hazard. The exoskeleton TC joint can be moved upwards or downwards to avoid the medial and lateral malleoli of the user. The exoskeleton joints are typically an area of bulk, so it can be advantageous to place these joints away from the user's malleoli, a physiological area of protrusion.

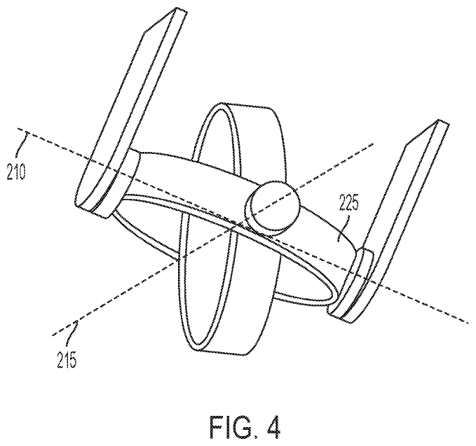

The exoskeleton 200 can be a gimbal structure. The exoskeleton 200 can include a linkage portion 205. The linkage portion 205 can include a first axis of rotation 210. The first axis of rotation 210 can be aligned with the TC joint 105. The linkage portion 205 can include a second axis of rotation 215. The second axis of rotation 215 can be aligned with the ST joint 110. As shown in FIG. 4, among others, the linkage portion 205 can be inverted to allow a human foot to fit within the confines of the structure of the exoskeleton 200. The linkage portion 205 can couple with a foot plate 220. The linkage portion 205 can be in the inverted orientation when coupled with the foot plate 220. The linkage portion 205 can be resolved in the foot plate 220. The linkage portion 205 can include a ST-TC link structure 225. The exoskeleton 200 can include a shank structure 230. The shank structure 230 can couple the exoskeleton 200 with the human shank 115. A portion of the linkage portion 205 can project up to couple with shank structure 230.

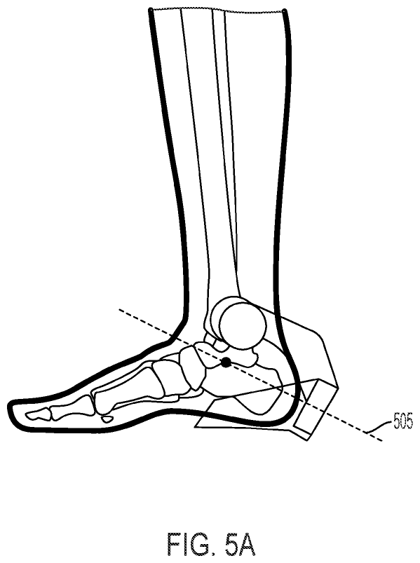

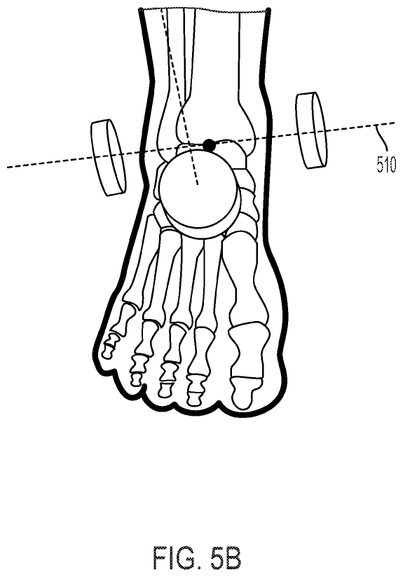

FIG. 5A illustrates a side view of a foot depicting a projection of a ST joint axis of rotation 505 with a mechanical joint posterior to the foot (shown on the sagittal plane).

FIG. 5B illustrates an anterior view of the foot depicting a projection of a TC joint axis of rotation 510 with mechanical joints co-linear to the TC joint 105.

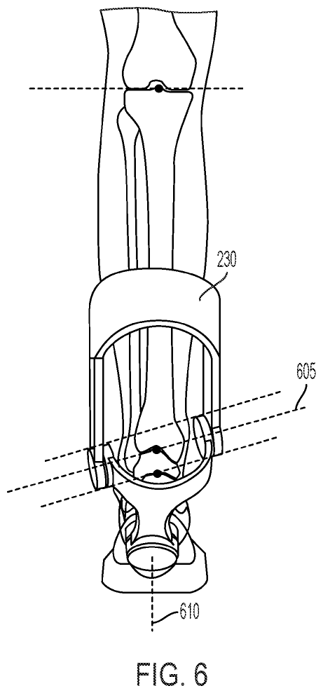

FIG. 6 illustrates a posterior view of the exoskeleton 200. The exoskeleton 200 can be aligned with a corresponding biological axis and human attachment points. The exoskeleton 200 can include a TC joint axis 605 that can align with the human TC joint 105. The exoskeleton 200 can include a ST joint axis 610 that can align with the human ST joint 110.

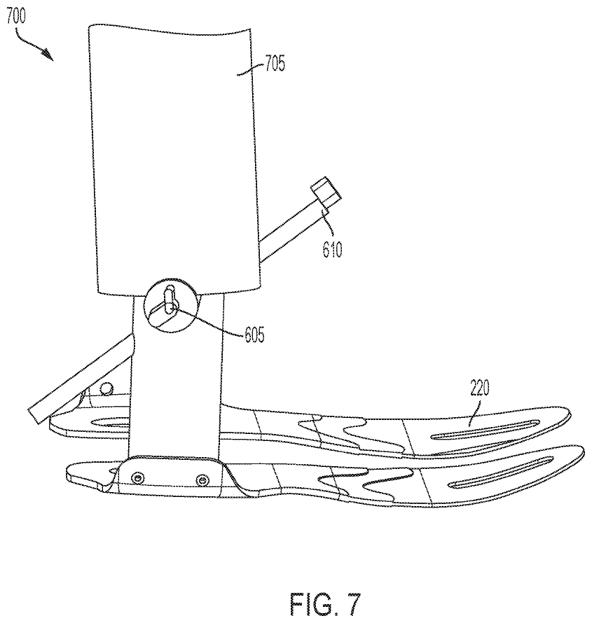

FIG. 7 illustrates an ankle model 700 showing relative joint axis and representative foot/leg volumes. The ankle model 700 can include the TC joint axis 605 and the ST joint axis 610. The ankle model 700 can include a human model structure 705. The human model structure 705 can include a human shank and ankle volumes. The ankle model 700 can include the foot plate 220. The foot plate 220 can be a carbon foot plate 220.

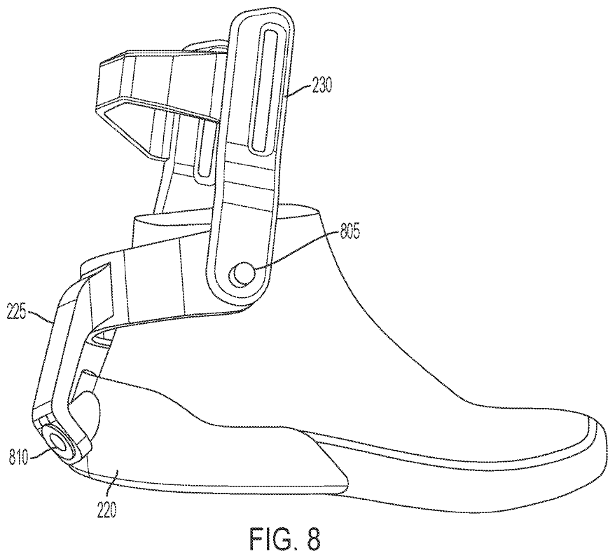

FIG. 8 illustrates a foot model 800 with an example exoskeleton 200. The foot model 800 is superimposed on the TC joint axis 605 and the ST joint axis 610 to build mechanical joint locations and structure relative to a human foot. The exoskeleton 200 of the foot model 800 can include a first mechanical joint 805. The first mechanical joint 805 can align with the TC joint 105. The exoskeleton 200 can include a second mechanical joint 810. The second mechanical joint 810 can align with the ST joint 110. The exoskeleton 200 can include the shank structure 230. The exoskeleton 200 can include the ST-TC link structure 225 to connect the first mechanical joint 805 with the second mechanical joint 810. The exoskeleton 200 can include a foot plate 220. The foot plate 220 can extend under and around a foot. The foot plate 220 can locate a lower half of the second mechanical joint 810 and translate force to the ground.

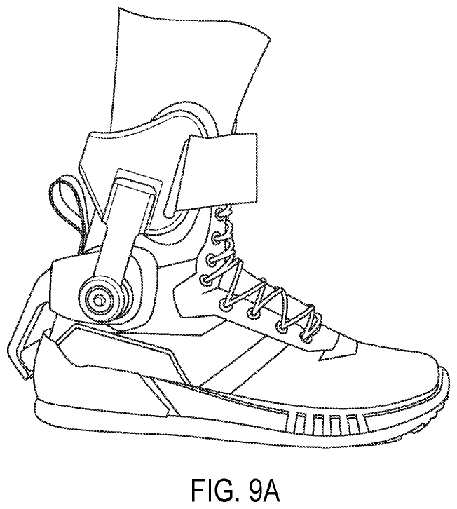

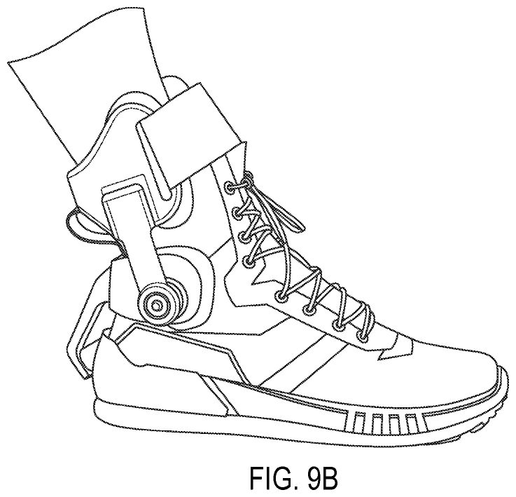

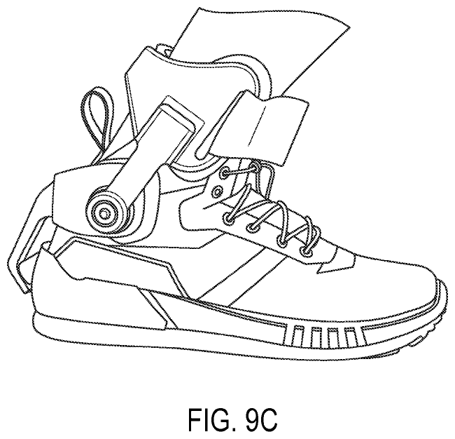

FIGS. 9A-9C illustrate the exoskeleton 200 integrated into footwear demonstrating plantarflexion and dorsiflexion degrees of freedom.

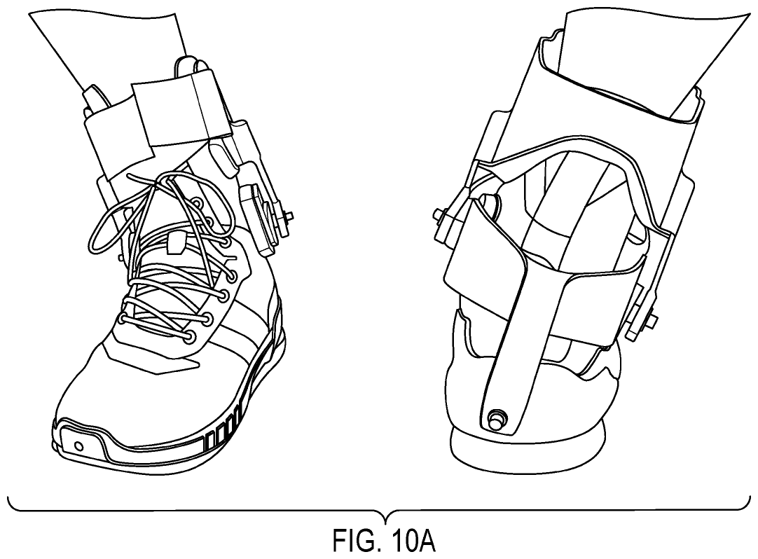

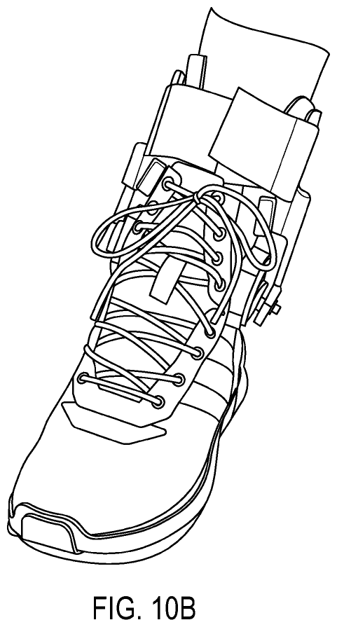

FIGS. 10A-10B illustrate the exoskeleton 200 integrated into footwear demonstrating inversion and eversion degrees of freedom.

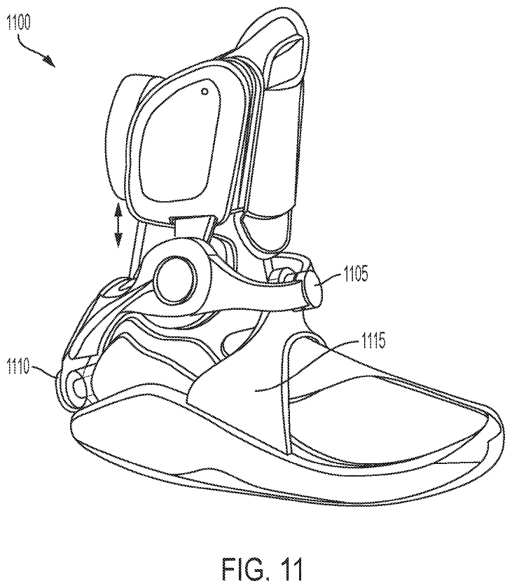

FIGS. 11-16 illustrate various embodiments of the exoskeleton 200. The number of exoskeleton joints used to constrain the TC and ST axes is an important consideration for manufacturability, donning, doffing, tripping, and comfort. As shown in FIG. 11, an exoskeleton can include both anterior and posterior exoskeleton ST joints along with medial and lateral exoskeleton TC joints. The thickness, spacing, and diameter of joints can all contribute to the loads and moments that a joint can withstand. Two collinear joints that are spaced further apart can withstand a greater moment than a single joint or two that are placed closer together. The use of multiple, spread-out joints can allow the exoskeleton to be constructed from lighter materials with lighter individual joints. For example, it may be possible to use plastic as the structure material instead of metal. This is both advantageous from a manufacturing perspective along with mass consideration.

Asymmetric joint designs can offer benefits in terms of bulk and usability, but their designs must be carefully considered to withstand augmentation loads and forces. An anterior-only or posterior-only ST joint is favorable for donning and doffing the device, but this single joint must withstand the full force of augmentation.

A variety of embodiments can be derived by using some or all parts of the exoskeleton 200 or “gimbal” structure in conjunction with known Human attachment and footwear construction techniques. By selectively removing or adding parts of the linkages that may either interfere with the foot or be advantageous for various design applications on and around the Human foot and ankle. For example, both the anterior and posterior sections can be integrated into footwear and those combined structures can in turn create several ankle exoskeleton embodiments. In general, the upper portion (above the TC joint) can be attached securely to the lower leg (shank) and the lower portion of the structure (below the TC joint) can encapsulate the foot. When force is applied between the two structures via a unidirectional actuator, the resulting ankle torque in reaction with the shin pad and ground can result in augmented plantarflexion.

Some embodiments can eliminate the anterior portion of the linkage and integrate the resulting structure into footwear with a conventional anterior entry and closure system around the foot and ankle.

In some embodiments, the mechanical ST joint can be placed in the anterior position on top of the foot. This can be done for certain packaging considerations such as leaving the rear of the structure open allowing for easy entry into an integrated soft goods footwear structure around the foot.

Some embodiments can be derived by varying the lateral symmetry. For example, the Medial side TC can be removed, and the resulting exoskeleton can still mechanically function as it would with either both lateral and medial TC joints and/or only a single lateral or medial TC joint given appropriate design and materials.

FIG. 11 illustrates an anterior and posterior ST joint integrated into footwear, with two ST mechanical joint locations, the force load can be distributed across more material allowing for increased comfort and less demand for materials with extreme strength to mass properties. FIG. 11 illustrates an example exoskeleton 1100. The exoskeleton 1100 can include an anterior mechanical joint 1105. The anterior mechanical joint 1105 can be aligned with the ST joint 110. The exoskeleton 1100 can include a posterior mechanical joint 1110. The posterior mechanical joint 1110 can be aligned with the ST joint 110. The exoskeleton 1100 can include a support structure 1115 to support the anterior mechanical joint 1105 and translate force to the ground.

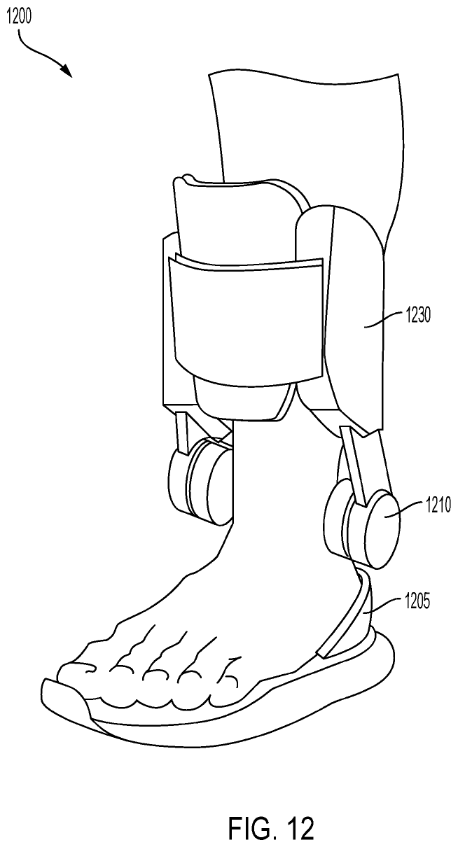

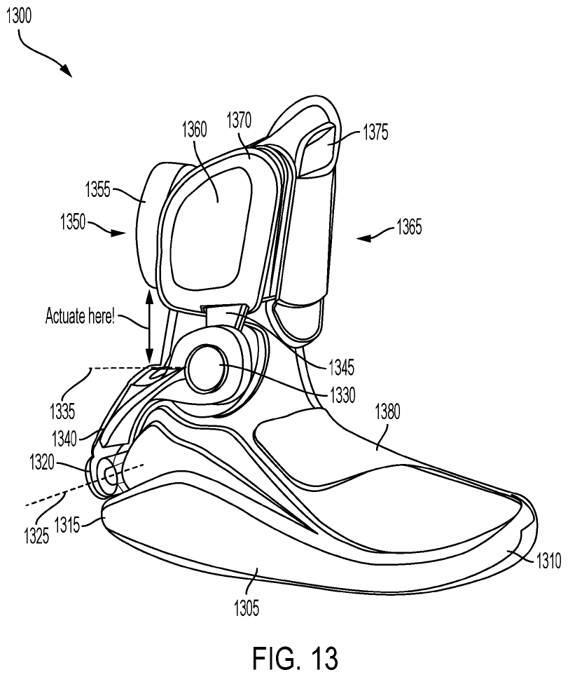

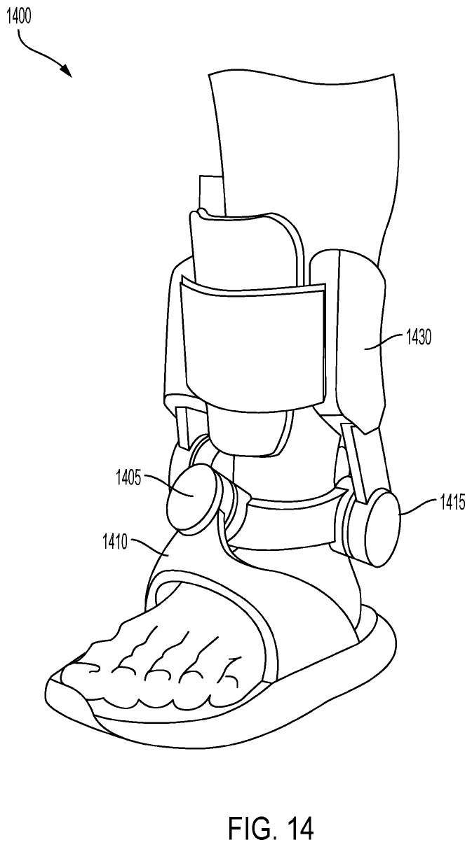

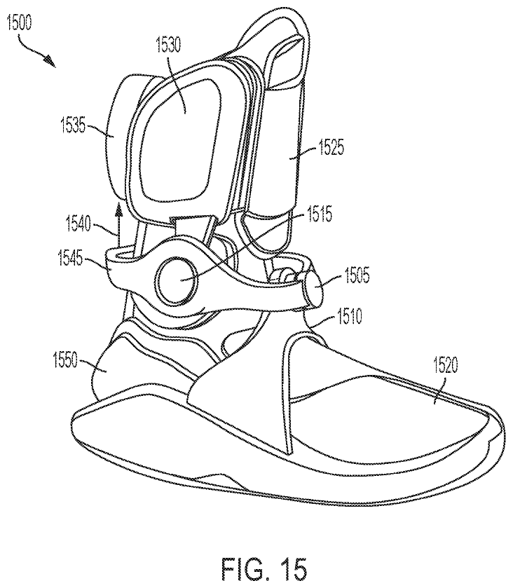

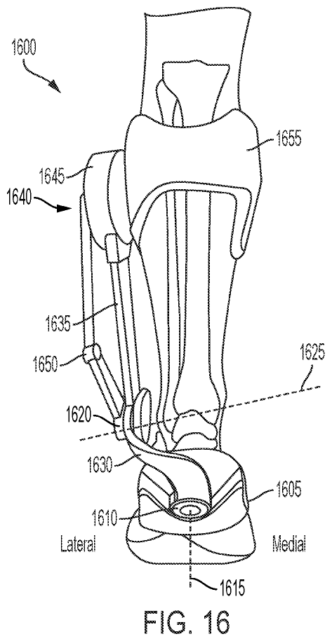

FIGS. 12-16 illustrate various exoskeleton embodiments. The embodiments can include asymmetrical joint structures. While a symmetrical joint structure can minimize joint and structure forces, it can present challenges for donning & doffing, fit, and bulk. Exoskeletal structures located on the medial side of the leg and foot can create an opportunity for the left and right devices to clip each other during use. This can be uncomfortable and potentially cause tripping. It is critical to minimize the amount that any device protrudes from the leg's natural medial surface. Furthermore, ST joints on both the anterior and posterior sides of the leg can pose a challenge to donning the device, especially if the joints are located closely to the body. FIGS. 12 and 13 illustrate exoskeletons that only include a posterior ST joint, and FIGS. 14 and 15 illustrate exoskeletons that only have an anterior ST joint. Since these joints do not have partners, they can be designed to withstand the augmentation torque applied at the TC joint. FIG. 16 illustrates an exoskeleton with a minimalistic design that only considers a lateral TC joint and a posterior ST joint. While both of these joints can be designed to withstand the augmentation moments, they can minimize the amount of bulk and promote easy donning and doffing.

FIG. 12 illustrates a bare structure of a posterior exoskeleton on the Human foot and ankle with only a posterior ST joint. The advantage of this configuration is the anterior portion of the foot and leg is left open for easy donning and doffing. Soft goods can be integrated into footwear. FIG. 12 illustrates an example exoskeleton 1200. The exoskeleton 1200 can include a first posterior mechanical joint 1205. The first posterior mechanical joint 1205 can be aligned with the ST joint 110. The exoskeleton 1200 can include a second posterior mechanical joint 1210. The second mechanical posterior joint 1210 can be located above the first posterior mechanical joint 1205. The exoskeleton 1200 can include a shank structure 1230. The shank structure 1230 can couple with the shank 115 and can contain electromechanical components.

FIG. 13 illustrates an exoskeleton 1300 with a posterior ST joint. The exoskeleton 1300 can include at least one foot plate 1305. The foot plate 1305 can be a part of footwear (e.g., a shoe, sneaker, or boot). The foot plate 1305 can be configured to interface with footwear or a lower limb (e.g., leg or foot) of a user. The foot plate 1305 can define the bottom of the exoskeleton 1300. The foot plate 1305 can have a front end 1310 and a back end 1315. The front end 1310 can be configured to be proximate to a toe of a user. The back end 1315 can be configured to be proximate to a heel of a user.

The exoskeleton 1300 can include at least one joint. For example, the exoskeleton 1300 can include at least one a first joint 1320. The first joint 1320 can be an ST joint. For example, the first joint 1320 can define a first axis 1325. The first axis 1325 can be an axis of rotation. The first axis 1325 can be configured to extend along a ST axis of the user. For example, the first joint 1320 can align with the ST joint 110 of the user. The first joint 1320 can be a posterior ST joint. For example, the first joint 1320 can be located at an end of the foot plate 1305. For example, the first joint 1320 can be located at a back end 1315 of the foot plate 1305. The first joint 1320 can be configured to be proximate to the heel of the user. The first joint 1320 can be located or positioned behind the heel of a user of the exoskeleton 1300. Having the first joint 1320 at the rear of the foot plate 1305 can correct imbalance and rotational inertia that can be present with the exoskeleton 1300. For example, the first joint 1320 can be a heavier component of the exoskeleton 1300, and having the heavier components posterior to the lower limb of the user can facilitate proper balance. Further, having the first joint 1320 at the rear can allow for a more natural alignment between the first joint 1320 and the ST joint 110 of the user, which can provide a more natural augmented motion and reduce relative movement between the exoskeleton 1300 and the user's lower limb.

The first joint 1320 can be mechanically coupled with the foot plate 1305. For example, the first joint 1320 can be directly or indirectly coupled with the foot plate 1305. For example, the first joint 1320 can be indirectly coupled with the foot plate 1305 via at least one intermediate member (e.g., at least one member or component extends between the first joint 1320 and the foot plate 1305). The first joint 1320 can be integrated in footwear. For example, footwear can include the foot plate 1305. The first joint 1320 can be integrally formed with the footwear.

The exoskeleton 1300 can include at least one second joint 1330. The second joint 1330 can be a TC joint. For example, the second joint 1330 can define a second axis 1335. The second axis 1335 can be an axis of rotation. The second axis 1335 can be configured to extend along a TC axis of the user. The second joint 1330 can be a lateral TC joint. For example, the second joint 1330 can be located at a side of the foot plate 1305. The second joint 1330 can be located on an outer side of the foot plate 1305 such that the second joint 1330 can be disposed on an outer (e.g., lateral) side of a lower limb of a user (e.g., on the side of a first lower limb that is facing away from a second lower limb). Aligning the first joint 1320 and the second joint 1330 with respective biological joints (e.g., the ST joint 110 and the TC joint 105) can facilitate more natural augmented motion and reduce movement between the exoskeleton 1300 and the user's lower limb, which can reduce discomfort and skin irritations.

The second joint 1330 can be mechanically coupled with the foot plate 1305. For example, the second joint 1330 can be mechanically coupled at a side of the foot plate 1305. The second joint 1330 can be directly or indirectly coupled with the foot plate 1305. For example, the second joint 1330 can be indirectly coupled with the foot plate 1305 via at least one intermediate member (e.g., at least one member or component can extend between the second joint 1330 and the foot plate 1305). The second joint 1330 can be integrated in footwear.

The exoskeleton 1300 can include at least one linkage member 1340. The linkage member 1340 can be coupled with the first joint 1320 and the second joint 1330. The linkage member 1340 can extend between the first joint 1320 and the second joint 1330. The linkage member 1340 can have a curved shape (e.g., have a radius of arc). For example, the linkage member 1340 can have a curved shape to extend around a portion of a lower limb of a user. The linkage member 1340 can extend around the heel of the user. The curved shape of the linkage member 1340 can provide some flexibility or springiness functionality to the exoskeleton 1300, wherein a straight linkage member 1340 can be rigid. Further, the curved shape of the linkage member 1340 can be less susceptible to fracturing or breaking such that the linkage member 1340 can tolerate a greater force without breaking.

The exoskeleton 1300 can include at least one structural member 1345. The structural member 1345 can provide support to the exoskeleton 1300. The structural member 1345 can extend from the second joint 1330. The structural member 1345 can extend along a lower limb of the user. For example, the structural member 1345 can extend vertically from second joint 1330. The structural member 1345 can extend along or be disposed or located on an outer (e.g., lateral) side of the lower limb.

The structural member 1345 can have a first end and a second end. The first end can be coupled with the second joint 1330. The second end can be coupled with an actuator system 1350 of the exoskeleton 1300. For example, the exoskeleton 1300 can have at least one actuator system 1350. The actuator system 1350 can exert a force on other components of the exoskeleton 1300 to augment motion of the user. For example, the actuator system 1350 can exert a force between the linkage member 1340 and the structural member 1345 to augment motion of the user. The actuator system 1350 can be a unidirectional actuator system 1350. For example, the actuator system 1350 can exert a force or resistance in a single direction. The unidirectional actuator system 1350 can provide augmented motion in a single direction and reduce unnecessary resistance to user motion in other directions.

The actuator system 1350 can include at least one actuator, shown as motor 1355. The motor 1355 can be at or proximate to the second end of the structural member 1345. The motor 1355 can be mechanically coupled with the structural member 1345. The motor 1355 can be disposed on a back side of the exoskeleton 1300. For example, the motor 1355 can be configured to be positioned on a posterior side of a lower limb of a user.

The actuator system 1350 can include at least one power source 1360. The power source 1360 can provide power to the motor 1355 to provide mechanical power to the exoskeleton 1300. The power source 1360 can be any type of local power source. For example, the power source 1360 can be a battery. The power source 1360 can be stored in any component of the exoskeleton 1300. For example, the power source 1360 can be stored in an ankle pad of the exoskeleton 1300.

The exoskeleton 1300 can include at least one brace member 1365. The brace member 1365 can be configured to couple the exoskeleton 1300 with a lower limb of a user. For example, the brace member 1365 can extend around at least a portion of the lower limb below a knee of the user. The brace member 1365 can be coupled with the structural member 1345. The brace member 1365 can include a plurality of components. For example, the brace member 1365 can include at least one ankle pad 1370. The ankle pad 1370 can be positioned on a lateral or medial side of the lower limb of the user. The ankle pad 1370 can contain or house the power source 1360.

The brace member 1365 can include at least one shin pad 1375. The shin pad 1375 can be positioned on an anterior side of the lower limb. The shin pad 1375 can be coupled with or be integral with the brace member 1365. The shin pad 1375 can move between a first (e.g., open) position and a second (e.g., closed) position to facilitate entry of the lower limb into the exoskeleton 1300 and securing the lower limb in the exoskeleton 1300.

The exoskeleton 1300 can include at least one strap 1380. The strap 1380 can move between a first (e.g., open) position and a second (e.g., closed) position to facilitate entry of the lower limb into the exoskeleton 1300 and securing the lower limb in the exoskeleton 1300. The strap 1380 can be configured to extend across a top of a foot of the user. For example, the strap 1380 can extend from a first side of the foot plate 1305 toward a second side of the foot plate 1305. The strap 1380 can be or include any material. For example, the strap 1380 can be rigid and include a harder material (e.g., plastic, metal). The strap 1380 can be flexible and include a more flexible material (e.g., nylon, polyester). The strap 1380 can include at least some elasticity. The strap 1380 can couple with the exoskeleton 1300 via a variety of mechanisms. For example, the strap 1380 can buckle, hook and loop, tie, snap, or adhere to the exoskeleton 1300, among others.

FIG. 14 illustrates an exoskeleton 1400 with an anterior ST joint only. The exoskeleton 1400 can include an anterior mechanical joint 1405. The anterior mechanical joint 1405 can align with the ST joint 110. The exoskeleton 1400 can include an anterior support structure 1410 to support the anterior mechanical joint 1405 and translate ground reaction forces. The exoskeleton 1400 can include a mechanical joint 1415. The mechanical joint can align with the TC joint 105. The exoskeleton 1400 can include a shank structure 1430. The shank structure 1430 can couple with the shank 115.

FIG. 15 illustrates an exoskeleton 1500 with an anterior only ST joint fully integrated into footwear. The exoskeleton 1500 can include an anterior mechanical joint 1505 aligned with a ST joint 110. The exoskeleton 1500 can include an anterior support structure 1510 to support the anterior mechanical joint 1505 and translate ground reaction forces. The exoskeleton 1500 can include a mechanical joint 1515 aligned with a TC joint 105. The exoskeleton 1500 can be fully integrated with footwear 1520 (e.g., a full shoe). The exoskeleton 1500 can include a shin pad 1525. The shin pad 1525 can be combined with a traditional shoe “tongue” constructions and frontal closure entry system. The exoskeleton 1500 can have a shank structure 1530. The shank structure 1530 can be disposed around the lateral and medial areas of the shank 115. The shank structure 1530 can store batteries, PCBs, or other components necessary for electro mechanical components of the exoskeleton 1500 to function. The exoskeleton 1500 can include a motor (e.g., electric motor) and winding mechanism 1535. The exoskeleton 1500 can include a tensile element 1540. The tensile element 1540 can be a belt, multiple filaments, or a combination of elements combined to create a flexible tensile structure. The exoskeleton 1500 can include a linkage 1545 to mechanically connect the anterior mechanical joint 1505 with the mechanical joint 1515. The exoskeleton 1500 can include a semi-rigid posterior structure 1550 to locate a heel of the foot within the footwear 1520.

FIG. 16 illustrates a posterior view of a lower limb (e.g., a lower leg, ankle, and foot) with an exoskeleton 1600. The exoskeleton 1600 can include at least one foot plate 1605. The foot plate 1605 can be a part of footwear (e.g., a shoe or boot structure). For example, the exoskeleton 1600 can include the footwear. The foot plate 1605 can be configured to interface with footwear or a lower limb (e.g., leg or foot) of a user. The foot plate 1605 can define the bottom of the exoskeleton 1600. The foot plate 1605 can have a front end and a back end. The front end 1310 can be configured to be proximate to a toe of a user. The back end can be configured to be proximate to a heel of a user.

The exoskeleton 1600 can include an asymmetric ST joint and linkage. For example, the exoskeleton 1600 can include at least one joint. For example, the exoskeleton 1600 can include at least one a first joint 1610. The first joint 1610 can be an ST joint. For example, the first joint 1610 can define a first axis 1615. The first axis 1615 can be an axis of rotation. The first axis 1615 can be configured to extend along a ST axis of the user. The first joint 1610 can be a posterior ST joint. For example, the first joint 1610 can be located at an end of the foot plate 1605. For example, the first joint 1610 can be located at a back end of the foot plate 1605. The first joint 1320 can be configured to be proximate to the heel of the user. Having the first joint 1610 at the rear of the foot plate 1605 can correct imbalance and rotational inertia that can be present with the exoskeleton 1600. For example, the first joint 1610 can be a heavier component of the exoskeleton 1600, and having the heavier components posterior to the lower limb of the user can facilitate proper balance. Further, having the first joint 1610 at the rear can allow for a more natural alignment between the first joint 1610 and the ST joint 110 of the user, which can provide a more natural augmented motion and reduce relative movement between the exoskeleton 1600 and the user's lower limb.

The first joint 1610 can be mechanically coupled with the foot plate 1605. For example, the first joint 1610 can be directly or indirectly coupled with the foot plate 1605. For example, the first joint 1610 can be indirectly coupled with the foot plate 1605 via at least one intermediate member (e.g., at least one member or component extends between the first joint 1610 and the foot plate 1605). The first joint 1610 can be integrated in footwear. For example, footwear can include the foot plate 1605. The first joint 1610 can be integrally formed with the footwear.

The exoskeleton 1600 can include at least one second joint 1620. The second joint 1620 can be a TC joint. For example, the second joint 1620 can define a second axis 1625. The second axis 1625 can be an axis of rotation. The second axis 1625 can be configured to extend along a TC axis of the user. The second joint 1620 can be a lateral TC joint. For example, the second joint 1620 can be located at a side of the foot plate 1605. The second joint 1620 can be located on an outer (e.g., lateral) side of the foot plate 1605 such that the second joint 1620 can be disposed on an outer (e.g., lateral) side of a lower limb of a user (e.g., on the side of a first lower limb that is facing away from a second lower limb). Aligning the first joint 1610 and the second joint 1620 with respective biological joints (e.g., the ST joint 110 and the TC joint 105) can facilitate more natural augmented motion and reduce movement between the exoskeleton 1600 and the user's lower limb, which can reduce discomfort and skin irritations.

The second joint 1620 can be mechanically coupled with the foot plate 1605. For example, the second joint 1620 can be mechanically coupled at a side of the foot plate 1605. The second joint 1620 can be directly or indirectly coupled with the foot plate 1605. For example, the second joint 1620 can be indirectly coupled with the foot plate 1605 via at least one intermediate member (e.g., at least one member or component can extend between the second joint 1620 and the foot plate 1605).

The exoskeleton 1600 can include at least one linkage member 1630. The linkage member 1630 can be coupled with the first joint 1610 and the second joint 1620. The linkage member 1630 can extend between the first joint 1610 and the second joint 1620. The linkage member 1630 can have a curved shape (e.g., have a radius of arc). For example, the linkage member 1630 can have a curved shape to extend around a portion of a lower limb of a user. The linkage member 1630 can extend around the lower limb to a lateral side of the lower limb. The curved shape of the linkage member 1630 can provide some flexibility or springiness functionality to the exoskeleton 1600, wherein a straight linkage member 1630 can be rigid. Further, the curved shape of the linkage member 1630 can be less susceptible to fracturing or breaking such that the linkage member 1630 can tolerate a greater force without breaking. The curved shape can allow the linkage member 1630 to wrap at least partially around the lower limb of the user from the heel to the lateral side of the lower limb such that both the first joint 1610 and the second joint 1620 can align with respective biological joints of the user (e.g., the ST joint 110 and the TC joint 105).

The exoskeleton 1600 can include at least one structural member 1635. The structural member 1635 can provide support to the exoskeleton 1300. The structural member 1635 can extend from the second joint 1620. The structural member 1635 can extend along a lower limb of the user. For example, the structural member 1635 can extend vertically from second joint 1620. The structural member 1635 can extend along or be disposed or located on an outer (e.g., lateral) side of the lower limb.

The structural member 1635 can have a first end and a second end. The first end can be coupled with the second joint 1620. The second end can be coupled with an actuator system 1640 of the exoskeleton 1600. For example, the exoskeleton 1600 can have at least one actuator system 1640. The actuator system 1640 can exert a force on other components of the exoskeleton 1600 to augment motion of the user. For example, the actuator system 1640 can exert a force between the linkage member 1630 and the structural member 1635 to augment motion of the user. The actuator system 1640 can be a unidirectional actuator system 1640. For example, the actuator system 1640 can exert a force or resistance in a single direction. The unidirectional actuator system 1640 can provide augmented motion in a single direction and reduce unnecessary resistance to user motion in other directions. The exoskeleton 1600 can be laterally actuated. For example, the actuator system 1640 can be a lateral actuator system 1640. The lateral actuator system 1640 can exert a lateral force on the lower limb of the user.

The structural member 1635 and the actuator system 1640 can be configured to be located on a side of the lower limb of the user. For example, the structural member 1635 and the actuator system 1640 can be disposed on the lateral side of the lower limb.

The actuator system 1640 can include at least one actuator, shown as motor 1645. The motor 1645 can be at or proximate to the second end of the structural member 1345. The motor 1645 can be mechanically coupled with the structural member 1635. The motor 1645 can be disposed on a side of the exoskeleton 1600. For example, the motor 1645 can be configured to be positioned on a lateral side of a lower limb of a user.

The actuator system 1640 can include at least one power source. The power source can provide power to the motor 1645 to provide mechanical power to the exoskeleton 1600. The power source can be any type of local power source. For example, the power source can be a battery. The power source can be stored in any component of the exoskeleton 1600. For example, the power source can be a part of or be coupled with the motor 1645.

The actuator system 1640 can include at least one actuator member 1650. The actuator member 1650 can be coupled with the motor 1645. The actuator member 1650 can be coupled with the second joint 1620. The actuator member 1650 can extend between the motor 1645 and the second joint 1620. For example, the actuator member 1650 can extend between the motor 1645 and the first end of the structural member 1635. The actuator member 1650 can be positioned on a side of the lower limb of the user. For example, the actuator member 1650 can be disposed on a lateral side of the lower limb. The structural member 1635 can be located between the actuator member 1650 and the lower limb of the user.

The exoskeleton 1600 can include at least one brace member 1655. The brace member 1655 can be configured to couple the exoskeleton 1600 with a lower limb of a user. For example, the brace member 1655 can extend around at least a portion of the lower limb below a knee of the user. The brace member 1655 can be coupled with the structural member 1635. For example, the brace member 1655 can be coupled with the structural member 1635 via the motor 1645.

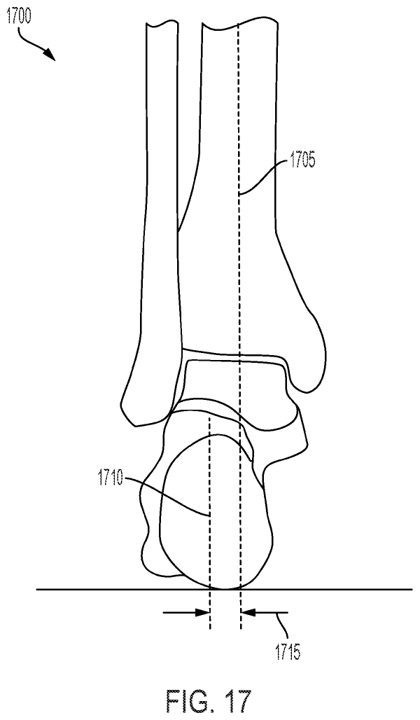

FIGS. 17-21 illustrate various ranges of motion and mechanical functions that enable movement of human joints. FIG. 17 illustrates a posterior view of a foot and ankle 1700 in a “normal” position showing ST joint alignment. Human joints actuated by the exoskeleton can have specific ranges of motion and mechanical functions that enable movement. To successfully augment these motions efficiently and without discomfort or injury, the ranges of motion and axis of rotation can be established, configured, customized or calibrated for a user. The foot and ankle 1700 can have a tibial mid diaphyseal line 1705, a calcaneal bisector line 1710, and a calcaneal-tibial angle 1715. The calcaneal-tibial angle 1715 can equal zero.

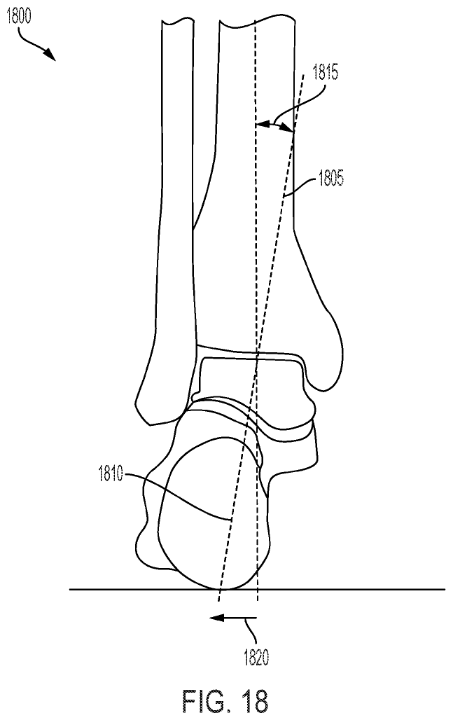

FIG. 18 illustrates a posterior view of a foot and ankle 1800 in a “valgus” position showing ST joint alignment. The foot and ankle 1800 can include a tibial mid diaphyseal line 1805, a calcaneal bisector line 1810, a calcaneal-tibial angle 1815, and a lateral translation 1820. The calcaneal-tibial angle 1815 can equal zero.

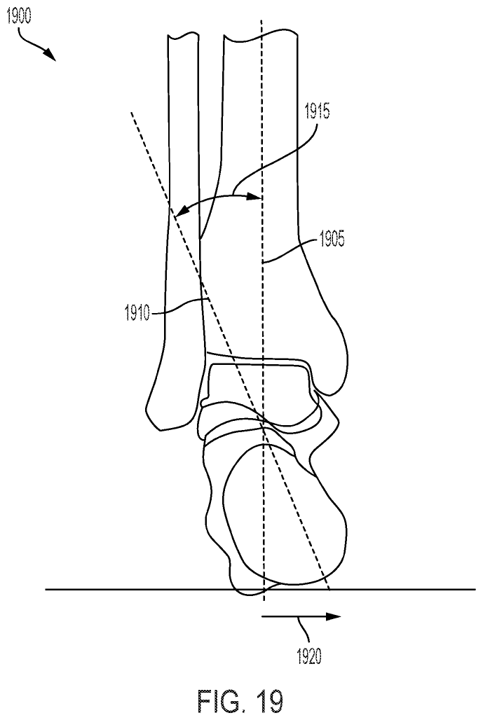

FIG. 19 illustrates a posterior view of a foot and ankle 1900 in a “Varus” position showing ST joint alignment. The foot and ankle 1900 can include a tibial mid diaphyseal line 1905, a calcaneal bisector line 1910, a calcaneal-tibial angle 1915, and a medial translation 1920. The calcaneal-tibial angle 1915 can equal thirty.

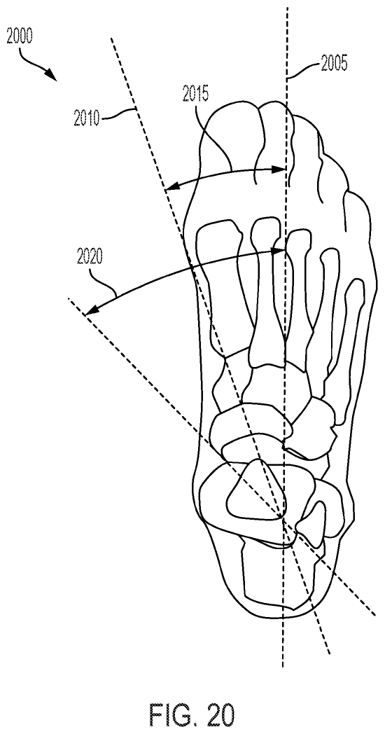

FIG. 20 illustrates a subtalar axis of rotation and ROM (transverse plane) of a foot and ankle 2000. The foot and ankle 2000 can have a sagittal plane 2005. A subtalar axis projection 2010 can be on a horizontal plane. A subtalar axis biological maxima 1 2015 can be in the horizontal plane. A subtalar axis biological maxima 2 2020 can be in the horizontal plane.

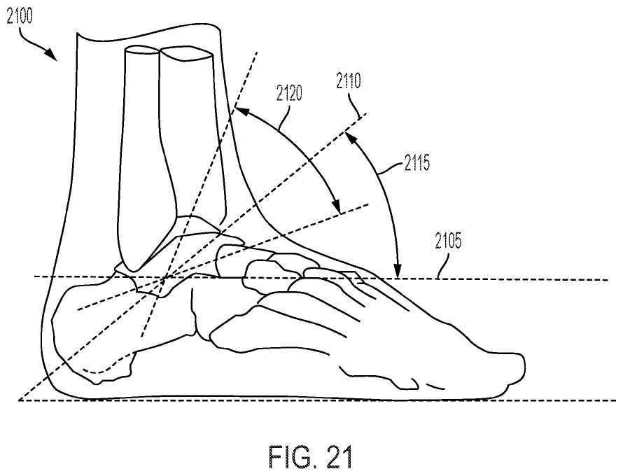

FIG. 21 illustrates a subtalar axis of rotation and ROM (Sagittal plane) of a foot and ankle 2100. The foot and ankle 2100 an have a horizontal plane 2105. A subtalar axis projection 2110 can be on a sagittal plane. The foot and ankle 2100 can have a subtalar axis biological maxima 1 2115 and a subtalar axis biological maxima 2120.

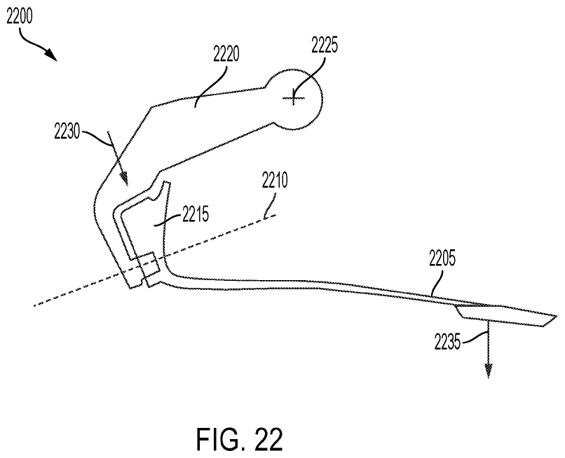

FIGS. 22-30 illustrate various embodiments of exoskeletons. Various embodiments can address mechanical function and quantifiable performance. Some additional considerations can include solving for cost so more individuals can have access, or manufacturability, so the device or product can be produced easily without specialized materials and machinery.

Some embodiments place the ST joint shaft under tension during plantar flexion augmentation and the structures above the ST joint under compression. This compression force can be translated through the talocrural-to-subtalar linkage, to the ground reaction structure built around the foot below it. Building in translational features designed to take force loads can reduce the need for highly engineered, exotic, and expensive materials. Designs requiring metal or high strength parts can be expensive, dense, and can cause irritation or harm to the wearer. It is favorable if a design can be realized and function appropriately with plastics and other lower cost and mass production friendly materials. An additional advantage inherent to the biomechanics of the device is that the joints do not have to rotate in a complete 360 degree arcs, therefore hard stops and a limited ROM can be incorporated to increase the strength of the parts.

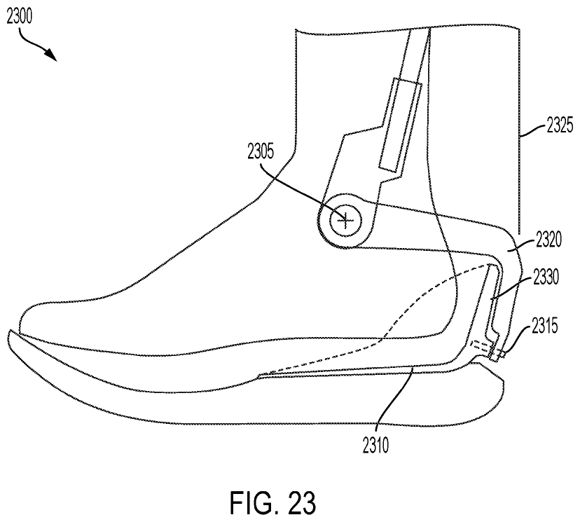

FIGS. 22 and 23 illustrate an example design that uses the limited range of motion of the ST joint to create a large bearing surface without the need for a large bearing. A simple bearing, pin, or screw can be used at the ST axis because a bearing surface is provided at a distance. This is possible because the ST joint can undergo less than 45 degrees of motion, so it does not require a fully circular bearing surface, and because augmentation torques can only be applied in the plantarflexion direction, so a compressive bearing surface can be used to react the moment in this direction. The combination of a simple pin joint and a displaced bearing surface can allow for the exoskeleton ST joint to be designed more closely to the human body in a lighter package.

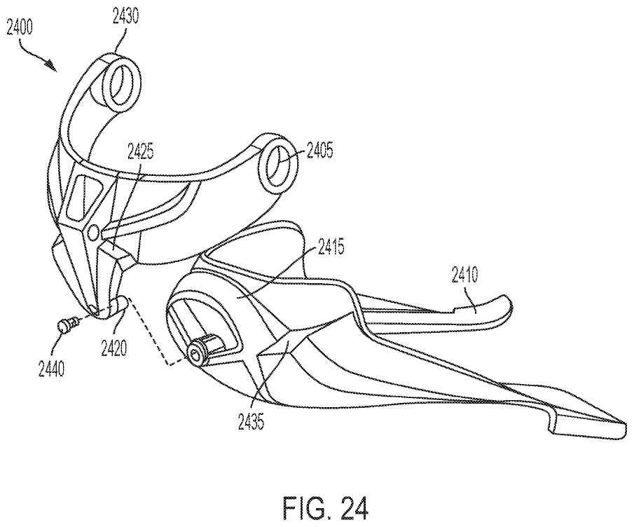

The limited range of eversion/inversion can also be protected by incorporating a hard stop in the exoskeleton ST joint. FIG. 24 illustrates how features in the foot structure and ST-TC Link structure can be nested to provide natural hard stops with the device reaches physiologically inspired limits in the range of motion.

FIG. 22 illustrates a cross-sectional view of an exoskeleton 2200 with a translative ST joint design. The exoskeleton 2200 can include a ground reaction structure 2205 that translates force and locates a lower half of an ST mechanical joint. The exoskeleton 2200 can include an axis of the ST mechanical joint 2210 and a mechanical axle designed to be under tension during plantarflexion augmentation. The exoskeleton 2200 can include a first feature 2215 built into the ground reaction structure 2205 to translate force from the TC-ST linkage 2220. The exoskeleton 2200 can include a TC mechanical joint 2225. The TC-ST linkage 2220 can connect the lower portion of the mechanical structure from a singular position (ST mechanical joint) behind the heel of the foot and project up to connect to two coincident positions aligned with the TC mechanical joints on either side of the ankle. The exoskeleton 2200 can include a second feature 2230 built into the TC-ST linkage 2220 that translates force from the TC-ST linkage 2220 to the ground reaction structure 2205 below it. The exoskeleton 2200 can receive or produce a ground reaction force 2235.

FIG. 23 illustrates a sagittal view of an exoskeleton 2300 with a translative ST joint design. The exoskeleton 2300 can include a TC mechanical joint 2305. The exoskeleton 2300 can include a ground reaction structure 2310 that translates force and locates a lower half of a ST mechanical joint. The exoskeleton 2300 can include an axis 2315 of the ST mechanical joint and mechanical axle designed to be under tension during plantarflexion augmentation. The exoskeleton 2300 can include a TC-ST linkage 2320 that connects the lower portion of the mechanical structure from a singular position (ST mechanical joint) behind the heel of the foot and project up to connect to two coincident positions aligned with the TC mechanical joints on either side of the ankle. The exoskeleton 2300 can include a tensile element 2325. The exoskeleton 2300 can include a sliding vertical surface 2330 that translates force from the ground reaction structure 2310 to the TC-ST linkage 2320.

FIG. 24 illustrates an exoskeleton 2400 with a translational ST joint assembly with a discrete range of motion built into the assembly. The exoskeleton 2400 can include a lateral TC joint location 2405. The exoskeleton 2400 can include a ground reaction structure 2410 that translates force and locates lower half of the ST joint. The exoskeleton 2400 can include a first sliding surface 2415 that translates force from the ground reaction structure 2410 to the TC-ST linkage structure. The exoskeleton 2400 can include an axis 2420 of ST joint and mechanical axle designed to be under tension during plantarflexion augmentation. The exoskeleton 2400 can include a second sliding surface 2425 that translates force from the TC-ST linkage structure to the ground reaction structure 2410. The exoskeleton 2400 can include a medial TC joint location 2430. The exoskeleton 2400 can include hard stops 2435 built into either end of the ROM. The exoskeleton 2400 can include a ST joint pin 2440.

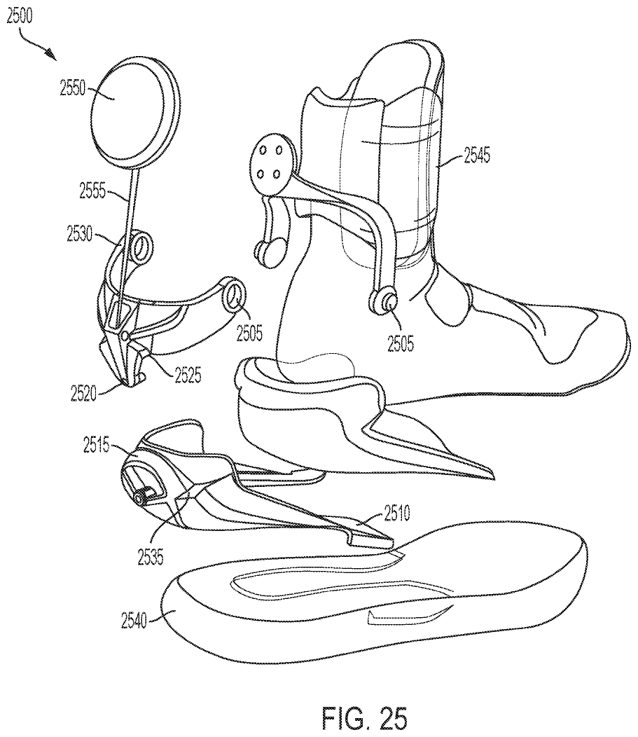

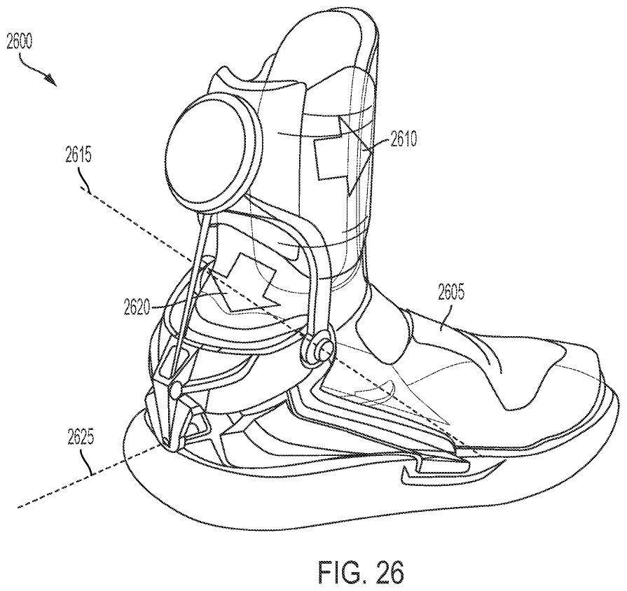

Along with jointed structures, an augmentation exoskeleton can require an actuator to apply augmenting moments to the user. An actuator may be as simple as a spring, or as complicated as a fully controllable electric motor and transmission. FIGS. 25 and 26 illustrate an exoskeleton system that implements a simple winch actuator, where a controllable motor is used to wind up a tensile element such as a cord or belt. The tensile element is connected to ST-TC Link structure, whereas the motor is connected to the shank structure. This allows for the heavy motor to be located more proximally on the body. Winding the tensile member exerts a force between the shank structure and TC-ST Link structure, resulting in a moment across the TC joint.

FIG. 25 illustrates an exploded view of an exoskeleton 2500 with a posterior ST joint and posterior spool actuator integrated into a singular article of footwear. The exoskeleton 2500 can include lateral TC joint location 2505. The exoskeleton 2500 can include a ground reaction structure 2510 that translates force and locates a lower half of the ST joint. The exoskeleton 2500 can include a first sliding surface 2515 that translates force from the ground reaction structure 2510 to the TC-ST linkage structure. The exoskeleton 2500 can include an axis 2520 of ST joint and mechanical axle designed to be under tension during plantarflexion augmentation. The exoskeleton 2500 can include a second sliding surface 2525 that translates force from the TC-ST linkage structure to the ground reaction structure 2510. The exoskeleton 2500 can include a medial TC joint location 2530. The exoskeleton 2500 can include hard stops 2535 built into either end of the ROM. The exoskeleton 2500 can include a shoe sole 2540. The exoskeleton 2500 can include a shoe upper and soft goods structure and electronics components 2545. The exoskeleton 2500 can include a motor and winch mechanism 2550. The exoskeleton 2500 can include a tensile element 2555. The structure that includes the ST bearing surface (first sliding surface 2515) and the ST hard stops 2535 is embedded between the lower midsole and either the upper midsole or a heel cup. A plate (not depicted) can be attached to this component that runs the partial or full length of the foot to transmit force to the ground.

FIG. 26 illustrates an exoskeleton 2600. The exoskeleton 2600 can be fully assembled and integrated into soft good footwear components and compliant materials around the foot. Higher mass items such as batteries and motors can be tightly affixed to the footwear without mechanical translation and skin shear during plantarflexion/dorsiflexion due to the ST joint being closer aligned to the natural Human ST joint. Elements under compression such as the structure supporting the winch and mechanical joints are ideally rigid however elements under tension can be compliant and soft to the touch if they have strong tensile properties. This allows for more comfortable, lighter weight constructions that are easier to get in and out of. The exoskeleton 2600 can include a shoe upper and soft goods structure and electronics components 2605. The exoskeleton 2600 can include an upper material 2610 under tensile load. The exoskeleton 2600 can include a TC joint axis 2615. The exoskeleton 2600 can include a structure 2620 resisting the winch under a compressive load. The exoskeleton 2600 can include a ST joint axis and axle 2625.

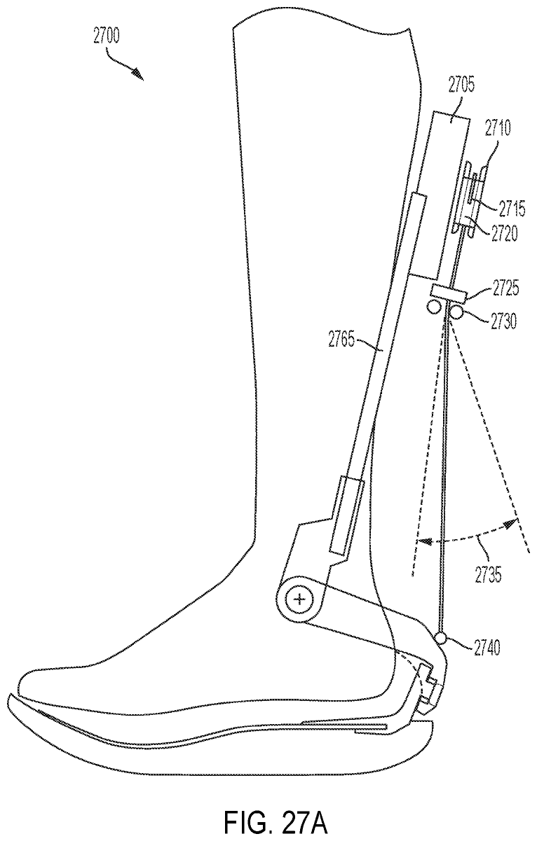

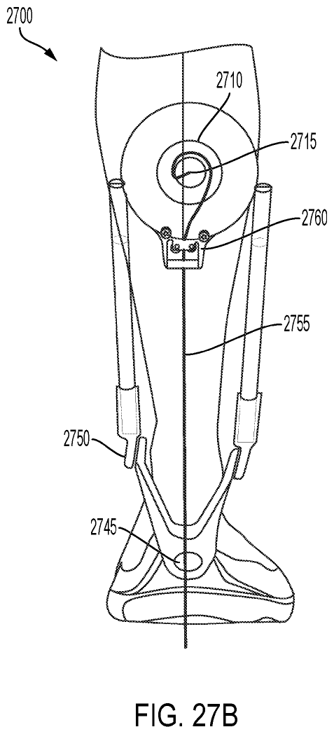

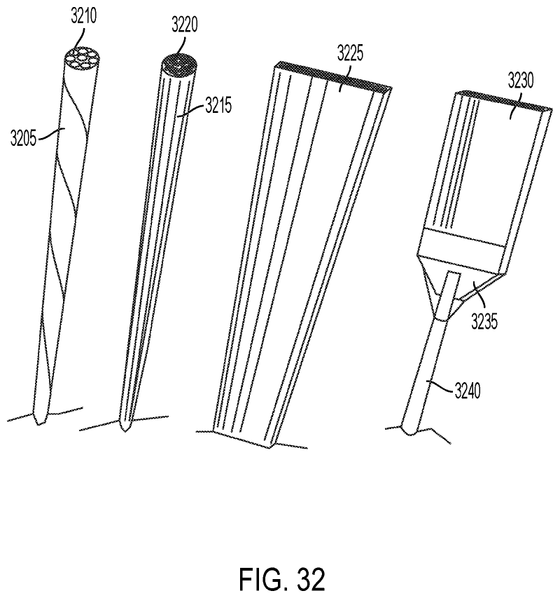

FIGS. 27A-32 illustrate various embodiments of exoskeletons, or portions thereof. The exoskeletons can include a tensile element. The tensile element must be carefully considered since it may be actuating a TC joint that is not orthogonal to the motor axis. The tensile element must be appropriately directed so that the actuator winch can consistently wrap the element. A series of idler pulleys or guides can be used to direct the tensile element in multiple directions (FIGS. 27-31). A circular cord is simple to guide but faces cycle limitations since the outermost fibers are under more tension than the innermost fibers. For this reason, a belt is favorable for the winch actuator, but it is difficult to redirect a belt in multiple directions since it is both flexible and rigid. FIG. 32 describes a hybrid solution where the tensile element is comprised of both a belt and a circular cord. The flat belt is used on the side that engages the winch, whereas the cord is used afterward to redirect the tensile element into a favorable engagement.

Another consideration specific to ankle exoskeleton designs that have a high degree of integration into the footwear is that the electromechanical armature portion of the exoskeleton should be very durable and will most likely remain considerably more expensive than the footwear component for the near future. Additionally, footwear is generally considered a wear item due to the breakdown and general cyclic fatigue of foam cushioning materials and abrasion related to frictional contact with the walk surface of the shoe sole. Because of this, parts or all of the actuator module could be used on multiple shoes.

Additional components specific to the ankle exoskeleton integrated into the shoe sole portion such as the carbon foot plate may also have an effective lifespan that is shorter than the rest of the components due to the extreme design requirements. The carbon plate must live under the foot and generally have enough compliance to deflect easily during unpowered flexion and normal wear however, the structure must also be stiff enough to manage the combined force loads of both vigorous movements that can generate peak forces up to 2-3 times body weight in addition to the torque produced from augmentation forces reacting through the carbon plate. Due to packaging related to shoe integration and the overall desire to reduce distill mass and non-cushioning profile thickness underfoot whenever possible, the plate structure will almost always be a compromise between the thinnest, lightest possible structure and a reasonable service life.

The soft goods components used in the shoe uppers typically are comprised of textile, non-woven and low-density foam materials that can get dirty, stretch, tear, and suffer abrasion with normal use. This suggests that the “shoe” component of the exoskeleton might have a more frequent replacement schedule as most the material and assembly techniques used in footwear are not consumer repairable or rebuildable.

FIGS. 27A-27B illustrate an exoskeleton 2700. The exoskeleton 2700 can include a winder mechanism for cord-type tensile element. The winder mechanism can include a pulley and flanges to retain a cord that wrap multiple times around a spool. One end of the cord can terminate in a capstan feature in the pulley and the other end of the cord can terminate in a swivel mechanism attached to the TC-ST joint linkage. Because the angle of the cord relative to the spool pulley changes during plantarflexion/dorsiflexion and inversion/eversion movements, the cord can travel through 2 sets of symmetrically opposed rollers to constrain each degree of freedom. The first set of rollers closest to the spool can align the cord to the pulley/spool and the second set of rollers can manage the angular difference of the cord throughout the ROM of the TC joint resulting from plantarflexion/dorsiflexion. This method can work with a cord because the cord cross-section is symmetrical and will react predictably against the rollers in either DOF unlike a planar structure such as a belt.

During plantarflexion and dorsiflexion, the angle of the tensile element (TE.) can change relative to the winding spool as can inversion and eversion. To manage the angle of the tensile element (TE.), roller guides can be placed on either side, and in both degrees of freedom.

A variety of flexible tensile elements and variations thereof are favorable for unidirectional actuators. In the use case described for an ankle exoskeleton actuated with a winch actuator, cord structures with a round cross-section and belts with planar cross-sections can be used, each with both advantages and disadvantages. Cord structures can be easy to guide through more complex paths due to their cross-sectional symmetry but are ultimately limited in cyclic functions as the individual fibers within the cord structure rub against each other in an unconstrained fashion and fail. Another disadvantage of cords is as they can wrap around a pulley and the layers begin to overlap, the effective pulley diameter changes effecting transmission ratios. Belts, or planar tensile structures can solve the problem of changing transmission ratios because they can be very thin and can overlap multiple times without dramatically effecting transmission ratio. Another advantage belts or planar structures have over cords, is by aligning all of the fibers on a single plane each fiber can be isolated from rubbing on the neighboring fibers and large force loads can be distributed across all fibers without damage. One disadvantage of belts is due to their planar shape they can only be constrained by rollers or guides in one DOF making routing paths and guides more complicated. A solution to this problem can be a hybrid tensile structure with a planar section designed specifically to wrap around a pulley that transitions into a cord structure with a round cross-section to run through guides and actuate mechanical components.

The exoskeleton 2700 can include a cord type tensile element and winding mechanism with 2 degrees of freedom roller guides. The exoskeleton 2700 can include a motor housing 2705. The exoskeleton 2700 can include a spool pulley flange 2710. The exoskeleton 2700 can include a spool pulley capstan 2715. The exoskeleton 2700 can include a spool pulley 2720. The exoskeleton 2700 can include upper roller guides 2725. The exoskeleton 2700 can include lower roller guides 2730. The exoskeleton 2700 can include an angular range 2735 of the cord during plantarflexion/dorsiflexion. The exoskeleton 2700 can include a swivel mechanism 2740 with at least 2 DOF (anchors cord). The exoskeleton 2700 can include a ST mechanical joint 2745. The exoskeleton 2700 can include a TC mechanical joint 2750. The exoskeleton 2700 can include a cord-type tensile element 2755 (round cross-section). The exoskeleton 2700 can include an assembly 2760 containing 2 DOF roller guides and mounting bracket. The exoskeleton 2700 can include a winch support 2765.

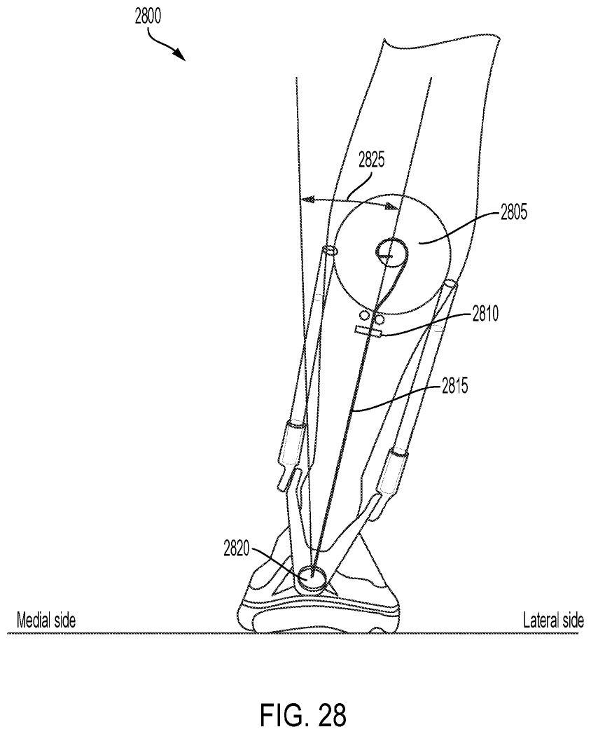

FIG. 28 illustrates an exoskeleton 2800 and a leg during eversion. The exoskeleton 2800 can include a winder mechanism 2805. The exoskeleton 2800 can include two DOF roller guides 2810. The exoskeleton 2800 can include a tensile element 2815. The exoskeleton 2800 can include a 2 DOF anchor 2820. The exoskeleton 2800 can include an angular ROM during eversion 2825.

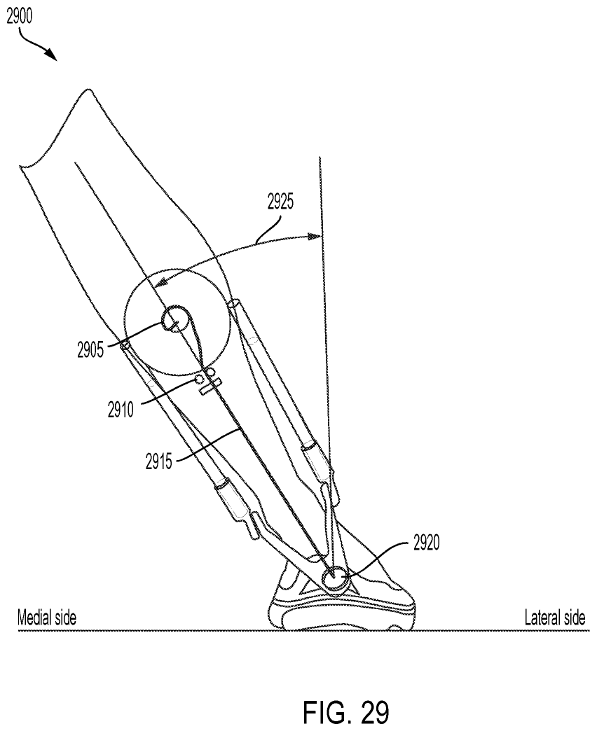

FIG. 29 illustrates an exoskeleton 2900 and a leg during inversion. The exoskeleton 2900 can include a winder mechanism 2905. The exoskeleton 2900 can include two DOF roller guides 2910. The exoskeleton 2900 can include a tensile element 2915. The exoskeleton 2900 can include a 2 DOF cord anchor 2920. The exoskeleton 2900 can include an angular ROM during inversion 2925.

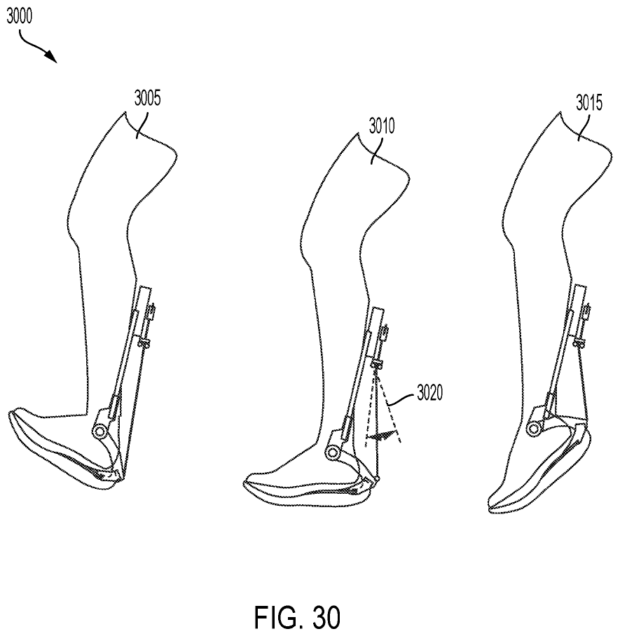

FIG. 30 illustrates an exoskeleton 3000 and a leg during plantarflexion/dorsiflexion showing the resulting angle of the tensile element during plantarflexion/dorsiflexion. Position 3005 illustrates the exoskeleton 3000 and the leg, ankle, and foot during dorsiflexion. Position 3010 illustrates the exoskeleton 3000 and the leg, ankle, and foot in a neutral stance. Position 3015 illustrates the exoskeleton 3000 and the leg, ankle, and foot during plantarflexion. FIG. 30 shows an angular range 3020 of the tensile element through dorsiflexion/plantarflexion.

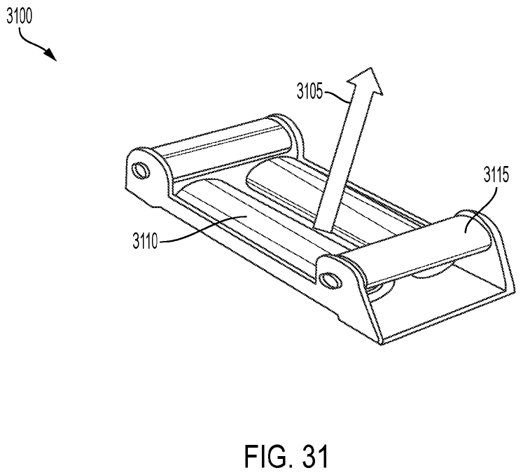

FIG. 31 illustrates a 2 DOF winch roller 3100 with parallel roller located on each axis. The 2 DOF winch roller 3100 can include a path 3105 of the tensile element. The 2 DOF winch roller 3100 can include a first set of rollers 3110 to constrain an angle of the tensile element. The 2 DOF winch roller 3100 can include a second set of rollers 3115 to align a cord path with a spool pulley.

FIG. 32 illustrates multiple embodiments of tensile elements, including round cross-sectional cord structures, planar belt-type structures, and a hybrid planar structure that transitions to a round cross-sectional cord structure. A first tensile cord structure 3205 can have a round cross-section and twisted bundles of fibers. Individual fibers 3210 can be grouped into bundles and packed into a larger diameter round cross-sectional cord structure. Second tensile cord structure 3215 can have a round cross-section and multiple aligned filaments. Multiple individual filaments 3220 are aligned within the cord diameter. A planar structure 3225 can be comprised of multiple filaments aligned in a parallel orientation without touching. Typically, a substrate material such as rubber or plastic can be used to constrain the fibers in the correct direction. A hybrid tensile structure 3230 can include a planar top section optimized for wrapping around a pulley. A first hybrid tensile structure 3235 can include a transitional area that can be fabricated as a single component or include a mechanical swivel transition combining the planar and round section into a singular tensile element. A second hybrid tensile structure 3240 can include a round cross-sectional area designed specifically to route easily through cable guides to actuators.

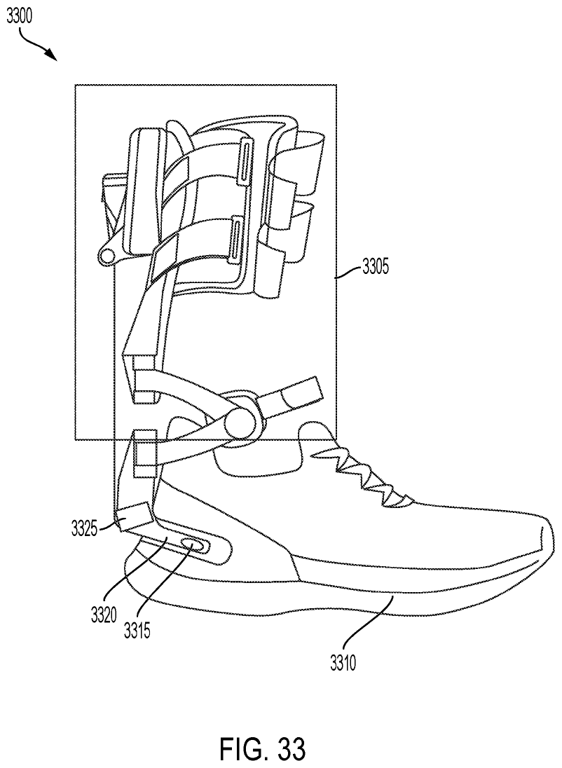

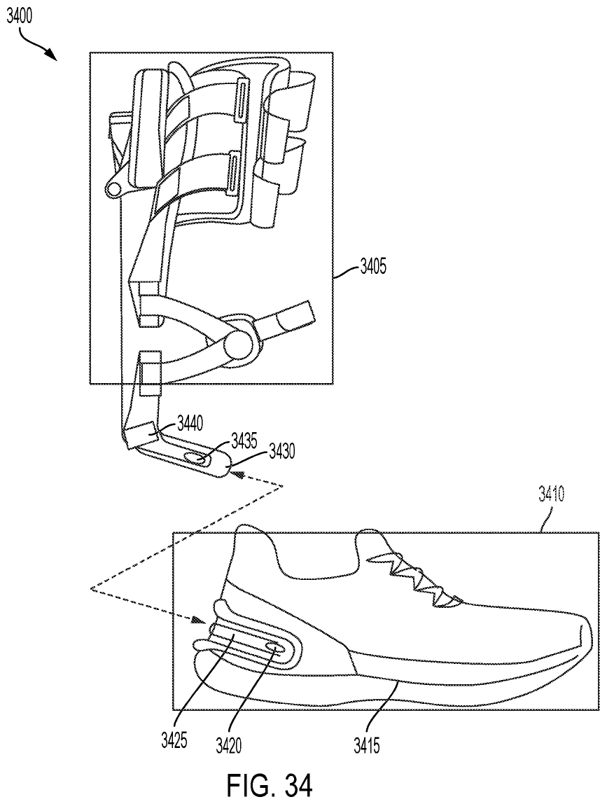

FIGS. 33 and 34 illustrate various embodiments of an exoskeleton with a quick disconnect/attachment mechanism. There can be numerous reasons to replace or swap the footwear component aside from just service life, such as specific performance attributes inherent to the materials and design related to different activities, aesthetic preferences, and/or comfort or considerations such as cold, hot or wet conditions. The soft-goods (shoe) component of the exoskeleton as shown in FIG. 33, among others, can be separated from the exoskeleton completely as shown in FIG. 34, with the mechanical armature of the exoskeleton and the soft-goods (shoe) component. In this embodiment, the interface between the two components employs a mechanical coupling that must be capable of transferring the force loads generated through movement and augmentation but engage and disengage easily and without special tools, excessive effort, or complex assembly. Because a moment of force must be transferred through the interface, the features locking the components together during use must be suitably robust and utilize enough surface area to maintain a secure connection during vigorous activity and high force loads.

It is also critical to the safety and function of the exoskeleton that the interface mechanism does not detach unintentionally during use. Discreet pushbutton-type mechanical locks on both the medial and lateral sides require both buttons to be depressed simultaneously to release. This simple 2-point redundancy to release the mechanism could prevent accidental decoupling during use by reducing the odds of a singular strike impacting a release. Tactile feel and audible feedback are also crucial to the design of the quick release mechanism to confirm to the user that the shoe component and the exoskeleton are securely connected. Other methods to disconnect and reconnect the exoskeleton and the footwear component quickly and securely would be to have a pin that serves the dual functions of both an axle the ST joint rotates about and a retaining pin. When installed the pin aligns and retains collinearity between a posterior shoe structure attachment point resolving in a hinge, and the TC-ST joint linkage of the exoskeleton resolving in a clevis. When the pin is removed, the two components are completely independent of each other. A push ball locking mechanism as commonly used in locking pins, or similar mechanical interference could be used to secure the pin in place during use and prevent unintentional uncoupling of the exoskeleton and footwear sections. The axle pin could also be completely retained within the TC-ST joint linkage of the exoskeleton to avoid losing or damaging the axle pin and component pieces during assembly and disassembly.

As shown above, FIG. 24 illustrates another opportunity for a quick-disconnect mechanism. The ST joint pin 2440 can be used to disconnect the electromechanical system from the footwear. This pin can implement a variety of mechanisms that allow a user to easily remove the pin. For example, the pin can implement simple threads, a quick-release ball pin, a locking twist cam, a magnetic ball lock, or a spring-loaded lock.

FIG. 33 illustrates an exoskeleton 3300 with a quick disconnect/attachment mechanism. The exoskeleton 3300 can include an electro-mechanical portion 3305 that can contain an upper shank mount, a rigid structure, a motor, a transmission, linkages, and other electrical and mechanical components terminated in a quick attach/disconnect posterior coupling such that it can be separated from the lower footwear component. The exoskeleton 3300 can include a shoe sole 3310 containing ground force reaction components, cushioning elements, and traction/wear surface. The exoskeleton 3300 can include a lateral release button 3315. This is a symmetrical design that has a mirrored medial release button that can be actuated simultaneously to release. The exoskeleton 3300 can include a posterior coupling 3320 that wraps around the heel of the shoe sole and rigidly connects to the ground reaction structures built into the shoe sole. The exoskeleton 3300 can include a mechanical ST joint 3325.

FIG. 34 illustrates an exoskeleton 3400 with a quick disconnect/attachment mechanism. The exoskeleton 3400 can include an electro-mechanical portion 3405 that can contain the upper shank mount, rigid structure, motor, transmission, linkages, and other electrical and mechanical components terminated in a quick attach/disconnect posterior coupling. The exoskeleton 3400 can couple with or be a part of a shoe 3410. The exoskeleton 3400 can include a shoe sole 3415 containing ground force reaction components, cushioning elements, and traction/wear surface. The exoskeleton 3400 can include a first lateral release button 3420. This is a symmetrical design that has a mirrored medial release button that can be actuated simultaneously to release. The exoskeleton 3400 can include a mechanical interface 3425 that receives and retains a posterior coupling that is both structurally integrated into the shoe, shoe upper and the ground reaction structures built into the shoe to comfortable translate force to the ground around the foot. The exoskeleton 3400 can include a posterior coupling 3430 that wraps around the heel of the shoe sole and rigidly connects to the ground reaction structures built into the shoe sole. The exoskeleton 3400 can include a second lateral release button 3435. This is a symmetrical design that has a mirrored medial release button that can be actuated simultaneously to release. The exoskeleton 3400 can include a mechanical ST joint 3440.

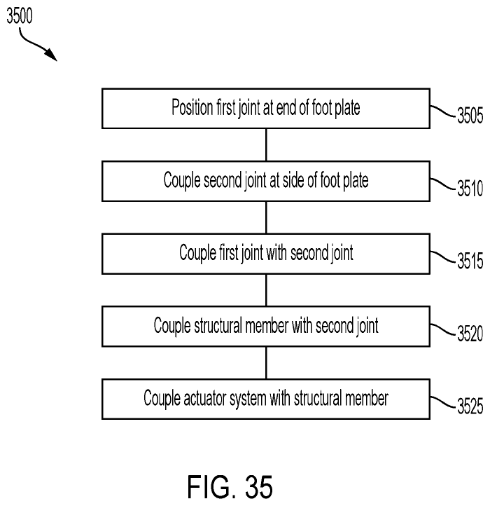

FIG. 35 illustrates a flow diagram of an example method 3500 of assembling an exoskeleton. As an illustrative example, method 3500 is described with reference to exoskeleton 1600, but method 3500 can be applied to other exoskeletons (e.g., exoskeleton 1300). Method 3500 can include positioning a first joint 1610 of the exoskeleton 1600 at an end of a foot plate 1605 (Act 3505). The first joint 1610 can be configured to be proximate to a heel of a user. The first joint 1610 can define a first axis 1615. Act 3505 can include positioning the first axis 1615 to align with a subtalar axis of a user.

Method 3500 can include coupling a second joint 1620 at a side of the foot plate 1605 (Act 3510). For example, the second joint 1620 can be positioned on a lateral side of the foot plate 1605. The second joint 1620 can define a second axis 1625. Act 3510 can include positioning the second axis 1625 to align with a talocrural axis of the user.

Method 3500 can include coupling the first joint 1610 with the second joint 1620 (Act 3515). For example, the first joint 1610 can be coupled with the second joint 1620 via a linkage member 1630.

Method 3500 can include coupling a structural member 1635 with the second joint 1620 (Act 3520). The structural member 1635 can extend along a lower limb of the user. The structural member 1635 can include or be coupled with a brace member 1655. The brace member 1655 can couple the exoskeleton 1600 with a lower limb of the user. Act 3520 can include positioning the brace member 1655 on a medial side of a structural member 1635 of the exoskeleton 1600. The brace member 1655 can extend around at least a portion of the lower limb of the user. The brace member 1655 can couple with the lower limb below a knee of the user.

Method 3500 can include coupling an actuator system 1640 with the structural member 1635 (Act 3525). The actuator system 1640 can exert a force between the linkage member 1630 and the structural member 1635 to augment motion of the user. Method 3500 can include coupling a first end of the structural member 1635 with the second joint 1620. Method 3500 can include coupling a second end of the structural member 1635 with a motor 1645 of the actuator system 1640. Method 3500 can include coupling an actuator member 1650 of the actuator system 1640 with the motor 1645 and the first end of the structural member 1635.

Method 3500 can include locating the structural member 1635 and the actuator system 1640, or at least a portion thereof, on a side of the foot plate 1605. The structural member 1635 can be located between the actuator member 1650 of the actuator system 1640 and the lower limb of the user.

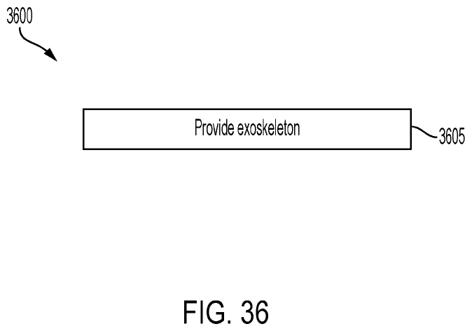

FIG. 36 illustrates a flow diagram of an example method 3600 for providing an exoskeleton. As an illustrative example, method 3600 is described with reference to exoskeleton 1600, but method 3600 can be applied to other exoskeletons (e.g., exoskeleton 1300). Method 3600 can include providing an exoskeleton 1600 (Act 3605). The exoskeleton 1600 can include a foot plate 1605. The exoskeleton 1600 can include a first joint 1610 located at an end of the foot plate. The first joint 1610 can define a first axis 1615. The first axis 1615 can extend along a subtalar axis of a user of the exoskeleton 1600. The first joint 1610 can be configured to be proximate to a heel of a user. The exoskeleton 1600 can include a second joint 1620. The second joint 1620 can be mechanically coupled at a side of the foot plate 1605. The second joint 1620 can define a second axis 1625. The second axis 1625 can extend along a talocrural axis of the user. The exoskeleton 1600 can include a linkage member 1630. The linkage member 1630 can be coupled with, and extend between, the first joint 1610 and the second joint 1620. The exoskeleton 1600 can include a structural member 1635. The structural member 1635 can extend from the second joint 1620. The structural member 1635 can extend along a lower limb of the user. The exoskeleton 1600 can include a brace member 1655. The brace member 1655 can be mechanically coupled with the structural member 1635. The brace member 1655 can couple the exoskeleton with the lower limb. The exoskeleton 1600 can include an actuator system 1640. The actuator system 1640 can be coupled with the structural member 1635. The actuator system 1640 can exert a force between the linkage member 1630 and the structural member 1635 to augment motion of the user.

Any references to implementations or elements or acts of the systems and methods herein referred to in the singular can include implementations including a plurality of these elements, and any references in plural to any implementation or element or act herein can include implementations including only a single element. References in the singular or plural form are not intended to limit the presently disclosed systems or methods, their components, acts, or elements to single or plural configurations. References to any act or element being based on any information, act or element may include implementations where the act or element is based at least in part on any information, act, or element.

Any implementation disclosed herein may be combined with any other implementation, and references to “an implementation,” “some implementations,” “an alternate implementation,” “various implementations,” “one implementation” or the like are not necessarily mutually exclusive and are intended to indicate that a particular feature, structure, or characteristic described in connection with the implementation may be included in at least one implementation. Such terms as used herein are not necessarily all referring to the same implementation. Any implementation may be combined with any other implementation, inclusively or exclusively, in any manner consistent with the aspects and implementations disclosed herein.

References to “or” may be construed as inclusive so that any terms described using “or” may indicate any of a single, more than one, and all of the described terms. References to at least one of a conjunctive list of terms may be construed as an inclusive OR to indicate any of a single, more than one, and all of the described terms. For example, a reference to “at least one of ‘A’ and ‘B’” can include only ‘A’, only ‘B’, as well as both ‘A’ and ‘B’. Elements other than ‘A’ and ‘B’ can also be included.

The systems and methods described herein may be embodied in other specific forms without departing from the characteristics thereof. The foregoing implementations are illustrative rather than limiting of the described systems and methods.

Where technical features in the drawings, detailed description or any claim are followed by reference signs, the reference signs have been included to increase the intelligibility of the drawings, detailed description, and claims. Accordingly, neither the reference signs nor their absence have any limiting effect on the scope of any claim elements.

The systems and methods described herein may be embodied in other specific forms without departing from the characteristics thereof. The foregoing implementations are illustrative rather than limiting of the described systems and methods. Scope of the systems and methods described herein is thus indicated by the appended claims, rather than the foregoing description, and changes that come within the meaning and range of equivalency of the claims are embraced therein.

What is claimed is:

1. An exoskeleton, comprising:
a foot plate;
a first joint located at an end of the foot plate, the first joint defining a first axis and configured to be proximate to a heel of a user;
a second joint mechanically coupled at a side of the foot plate, the second joint defining a second axis,
a linkage member coupled with, and extending between, the first joint and the second joint;
a structural member extending from the second joint, the structural member to extend along a lower limb of the user;
a brace member mechanically coupled with the structural member, the brace member to couple the exoskeleton with the lower limb; and
an actuator system coupled with the structural member, the actuator system to exert a force between the linkage member and the structural member to augment motion of the user.

2. The exoskeleton of claim 1, comprising:
the second joint to be located on a lateral side of the lower limb of the user.

3. The exoskeleton of claim 1, comprising:
the linkage member comprising a curved shape to extend around a portion of the lower limb of the user to a lateral side of the lower limb.

4. The exoskeleton of claim 1, comprising:
the first axis to extend along a subtalar axis of the user; and
the second axis to extend along a talocrural axis of the user.

5. The exoskeleton of claim 1, comprising:
the structural member comprising a first end and a second end, the first end coupled with the second joint; and
the actuator system, comprising:
a motor coupled with the second end and the structural member; and
an actuator member extending between the motor and the first end.

6. The exoskeleton of claim 1, comprising:
the structural member and the actuator system to be located on a side of the lower limb of the user; and
the actuator system comprising an actuator member, the structural member to be located between the actuator member and the lower limb of the user.

7. The exoskeleton of claim 1, comprising:
the actuator system comprising a motor, the motor to be disposed on a posterior side of the lower limb of the user.

8. The exoskeleton of claim 1, comprising:
the brace member to extend around at least a portion of the lower limb below a knee of the user.

9. The exoskeleton of claim 1, comprising:
the actuator system, comprising:
a motor; and
an actuator member coupled with the motor, the actuator member extends between the motor and the second joint, wherein the actuator system is a unidirectional actuator system.

10. The exoskeleton of claim 1, comprising:
the first joint integrated in footwear, the footwear comprising the foot plate.

11. The exoskeleton of claim 1, comprising:
a strap extending from a first side of the foot plate toward a second side of the foot plate, the strap to extend across a top of a foot of the user.

12. The exoskeleton of claim 1, comprising:
a shin pad to be positioned on an anterior side of the lower limb, the shin pad coupled with or integral with the brace member.

13. A method of assembling an exoskeleton, comprising:
positioning a first joint at an end of a foot plate, the first joint defining a first axis and configured to be proximate to a heel of a user;
coupling a second joint at a side of the foot plate, the second joint defining a second axis;
coupling the first joint with the second joint via a linkage member;
coupling a structural member with the second joint, the structural member to extend along a lower limb of the user, the structural member comprising a brace member to couple the exoskeleton with the lower limb; and
coupling an actuator system with the structural member, the actuator system to exert a force between the linkage member and the structural member to augment motion of the user.

14. The method of claim 13, comprising:
positioning the second joint on a lateral side of the foot plate.

15. The method of claim 13, comprising:
positioning the first axis to align with a subtalar axis of the user; and
positioning the second axis to align with a talocrural axis of the user.

16. The method of claim 13, comprising:
coupling a first end of the structural member with the second joint;
coupling a second end of the structural member with a motor of the actuator system; and
coupling an actuator member of the actuator system with the motor and the first end of the structural member.

17. The method of claim 13, comprising:
locating the structural member and the actuator system on the side of the foot plate, the structural member to be located between an actuator member of the actuator system and the lower limb of the user.

18. The method of claim 13, comprising:
positioning the brace member on a medial side of the structural member, the brace member to extend around at least a portion of the lower limb of the user.

19. A system configured to augment movement of a user, the system comprising:
an exoskeleton, comprising:
a foot plate;
a first joint located at an end of the foot plate, the first joint defining a first axis and configured to be proximate to a heel of the user;
a second joint mechanically coupled at a side of the foot plate, the second joint defining a second axis,
a linkage member coupled with, and extending between, the first joint and the second joint;
a structural member extending from the second joint, the structural member to extend along a lower limb of the user;
a brace member mechanically coupled with the structural member, the brace member to couple the exoskeleton with the lower limb; and
an actuator system coupled with the structural member, the actuator system to exert a force between the linkage member and the structural member to augment motion of the user.

20. The system of claim 19, comprising:
the first axis to extend along a subtalar axis of the user; and
the second axis to extend along a talocrural axis of the user.
